SEQUENCE LISTING (20 sequences)

SEQ ID NO: 1 - protein, 322 aa, Homo sapiens
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 2 - DNA, 966 bp, Homo sapiens
atgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaag

SEQ ID NO: 3 - protein, 354 aa, Homo sapiens
MSLRGSLSRLLQTRVHSILKKSVHSVAVIGAPFSQGQKRKGVEHGPAAIREAGLMKRLSSLGCHLKDFGDLSFTPVPKDDLYNNLIVNPRSVGLANQELAEVVSRAVSDGYSCVTLGGDHSLAIGTISGHARHCPDLCVVWVDAHADINTPLTTSSGNLHGQPVSFLLRELQDKVPQLPGFSWIKPCISSASIVYIGLRDVDPPEHFILKNYDIQYFSMRDIDRLGIQKVMERTFDLLIGKRQRPIHLSFDIDAFDPTLAPATGTPVVGGLTYREGMYIAEEIHNTGLLSALDLVEVNPQLATSEEEAKTTANLAVDVIASSFGQTREGGHIVYDQLPTPSSPDESENQARVRI

SEQ ID NO: 4 - DNA, 1080 bp, Homo sapiens
atgtctttaagaggctctttatctcgtctgctgcagactcgtgtgcacagcattttaaaaaagagcgtgcacagcgtggccgtcattggagcccccttcagccaaggccagaagagaaagggcgtcgaacacggacccgccgccatcagagaagctggtttaatgaagagactgagctctttaggctgccatttaaaagacttcggagatttgtcttttacccccgtccccaaggacgatttatacaataatttaatcgtgaaccccagatccgtgggactggctaaccaagaactggccgaggtcgtgagcagagccgtgtccgacggctactcttgtgtgactttaggcggcgatcactctttagccattggcacaatctccggacacgctaggcactgccccgatttatgcgtggtgtgggtggacgctcacgccgacatcaatacccctctgaccaccagcagcggcaatttacacggacagcccgtcagctttttactgagggagctgcaagataaggtgcctcagctgcccggcttcagctggatcaagccttgtatcagcagcgcttccatcgtgtacattggtttaagagacgtggaccctcccgaacacttcatcctcaagaactacgacattcagtacttcagcatgagggatatcgatcgtctcggaatccagaaggtgatggaaaggaccttcgatttactcatcggaaagaggcagaggcctatccatttatccttcgacatcgacgccttcgatcctacactggcccccgctactggtacacccgttgtgggcggtttaacctatagggagggcatgtacatcgccgaagagatccacaacaccggtttactgagcgctctggatttagtggaggtgaatcctcagctggccacctccgaggaggaggccaaaaccaccgccaatctggccgtggacgtgatcgccagctccttcggccagaccagagagggcggccacattgtgtacgaccagctgcccacacccagctcccccgatgagtccgaaaaccaagctcgtgtcagaatctgataataacccaagctt

SEQ ID NO: 5 - protein, 263 aa, Artificial Sequence
  synthetic construct
DILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGSGGGGSGGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSAKTTPPSVYGRVTVSS

SEQ ID NO: 6 - protein, 248 aa, Artificial Sequence
  synthetic construct
GSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGGGGSGGGGSSGGGSQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSS

SEQ ID NO: 7 - protein, 241 aa, Artificial Sequence
  synthetic construct
MQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGVGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIK

SEQ ID NO: 8 - protein, 221 aa, Artificial Sequence
  synthetic construct
QVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDY

SEQ ID NO: 9 - DNA, 786 bp, Artificial Sequence
  synthetic construct
atggactggatttggcgcatccttttccttgtcggcgctgctaccggcgcgcattctcaggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtacaagaggctattctagtacctcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctttaacgtgcctcttacattcggtgatggcaccaagcttgaaaaagctcta

SEQ ID NO: 10 - protein, 262 aa, Artificial Sequence
  synthetic construct
MDWIWRILFLVGAATGAHSQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEKAL

SEQ ID NO: 11 - protein, 42 aa, Artificial Sequence
  synthetic construct
KRGRKKLLYIFKQPFMRPVQTTQEEDGCSCRFPEEEEGGCEL

SEQ ID NO: 12 - protein, 36 aa, Artificial Sequence
  synthetic construct
RDQRLPPDAHKPPGGGSFRTPIQEEQADAHSTLAKI

SEQ ID NO: 13 - protein, 107 aa, Artificial Sequence
  synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 14 - protein, 38 aa, Artificial Sequence
  synthetic construct
CWLTKKKYSSSVHDPNGEYMFMRAVNTAKKSRLTDVTL

SEQ ID NO: 15 - protein, 112 aa, Artificial Sequence
  synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - DNA, 342 bp, Artificial Sequence
  synthetic construct
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 17 - protein, 1431 aa, Artificial Sequence
  synthetic construct
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGSGGGGSGGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSAKTTPPSVYGRVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 18 - DNA, 8755 bp, Artificial Sequence
  synthetic construct
ctcgagagctttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgctggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagttactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattagtactctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttggctaactagatctgtatctggcggtcccgcggaagaactgacgagttcgtattcccggccgcagcccctgggagacgtcccagcggcctcgggggcccgttttgtggcccattctgtatcagttaacctacccgagtcggactttttggagctccgccactgtccgaggggtacgtggctttgttgggggacgagagacagagacacttcccgcccccgtctgaatttttgctttcggttttacgccgaaaccgcgccgcgcgtcttgtctgctgcagcatcgttctgtgttgtctctgtctgactgtgtttctgtatttgtctgaaaattagctcgacaaagttaagtaatagtccctctctccaagctcacttacaggcggccgaattcgccgccgccatgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatccacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgaacggctggagctggagcctgtcgaccgagtgaagcagaccctgaactttgatctgctgaagctggccggcgacgtggagtccaaccccgggccagggaatatggccttaccagtgaccgccttgctcctgccgctggccttgctgctccacgccgccaggccggatattctgctcacacagaccccactctccctgcccgtgtcactcggggatcaggctagcatttcttgccgctcatctcagtctctggtccaccggaatgggaacacatacctccattggtacctccagaaacctggacagagccctaaactgctcatccacaaagtctcaaatcggttctccggcgtgcccgatcgctttagcggatccggatctgggaccgacttcacactgaaaatctcacgagtggaggctgaggatctcggcgtctacttctgtagtcagagtacccacgtcccacccctcacctttggcgctggaacaaaactggagctgaaacgagccgatgctgctcctaccgtgtccatctttcctggctccgggggaggcgggagcggaggcgaagtgaaactccagcagtctggcccttctctcgtggaacctggcgcttctgtgatgatctcctgtaaggcctctggatcttcctttaccggctacaacatgaactgggtccggcagaacattggcaaatccctggaatggattggcgccatcgatccttactacggcggcacatcatacaatcagaaattcaaggggcgagcaacactcactgtcgacaaatcttcatccaccgcctacatgcacctgaaatctctcacatccgaggatagtgctgtctactactgtgtctctggcatggaatactggggacagggaacttctgtcaccgtgtctagtgccaaaaccacacctccctccgtgtacggacgagtcactgtctcatctgctgaaccaaaatcctgtgacaaaacacacacatgcccaccttgtcctgcccctgaactgctcggcggaccttccgtctttctgtttccccccaaacccaaggatacactcatgatttctaggacccccgaagtcacttgtgtcgtggtcgatgtgtctcacgaggatcctgaagtgaaattcaactggtacgtggacggagtcgaggtccacaatgccaaaacaaaaccccgggaggaacagtacaatagcacctaccgagtcgtgtccgtgctcaccgtcctccatcaggattggctgaacggcaaagagtacaagtgtaaagtgagtaacaaggctctccccgctcctattgaaaaaaccatctcaaaagcaaaaggccagcctagggagcctcaggtctacacactgccaccctcacgggacgaactcaccaaaaatcaggtgtccctcacttgcctggtgaaaggcttctacccttccgatatcgctgtggaatgggagtcaaatgggcagcccgaaaacaactacaaaacaaccccccctgtgctcgattccgatggctcttttttcctgtactccaaactcaccgtggacaaatcacgctggcagcaggggaatgtcttttcttgctccgtgatgcacgaggccctccacaatcattacacccagaaatccctctcactctcacccggcaaaaaggaccctaaaaccacgacgccagcaccgcgaccaccaacaccggcgccaaccatcgcatcgcagcccctgtccctgcgcccagaggcgtgccgaccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggcagcggcgccaccaacttcagcctgctgaagcaggccggcgacgtggaggaaaaccctggccccatgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaataataaaagcttaacacgagccatagatagaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactgcccacctcgggggtctttcatt

SEQ ID NO: 19 - protein, 1463 aa, Artificial Sequence
  synthetic construct
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGSGGGGSGGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSAKTTPPSVYGRVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMSLRGSLSRLLQTRVHSILKKSVHSVAVIGAPFSQGQKRKGVEHGPAAIREAGLMKRLSSLGCHLKDFGDLSFTPVPKDDLYNNLIVNPRSVGLANQELAEVVSRAVSDGYSCVTLGGDHSLAIGTISGHARHCPDLCVVWVDAHADINTPLTTSSGNLHGQPVSFLLRELQDKVPQLPGFSWIKPCISSASIVYIGLRDVDPPEHFILKNYDIQYFSMRDIDRLGIQKVMERTFDLLIGKRQRPIHLSFDIDAFDPTLAPATGTPVVGGLTYREGMYIAEEIHNTGLLSALDLVEVNPQLATSEEEAKTTANLAVDVIASSFGQTREGGHIVYDQLPTPSSPDESENQARVRI

SEQ ID NO: 20 - DNA, 8854 bp, Artificial Sequence
  synthetic construct
ctcgagagctttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgctggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagttactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattagtactctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttggctaactagatctgtatctggcggtcccgcggaagaactgacgagttcgtattcccggccgcagcccctgggagacgtcccagcggcctcgggggcccgttttgtggcccattctgtatcagttaacctacccgagtcggactttttggagctccgccactgtccgaggggtacgtggctttgttgggggacgagagacagagacacttcccgcccccgtctgaatttttgctttcggttttacgccgaaaccgcgccgcgcgtcttgtctgctgcagcatcgttctgtgttgtctctgtctgactgtgtttctgtatttgtctgaaaattagctcgacaaagttaagtaatagtccctctctccaagctcacttacaggcggccgaattcgccgccgccatgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatccacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgaacggctggagctggagcctgtcgaccgagtgaagcagaccctgaactttgatctgctgaagctggccggcgacgtggagtccaaccccgggccagggaatatggccttaccagtgaccgccttgctcctgccgctggccttgctgctccacgccgccaggccggatattctgctcacacagaccccactctccctgcccgtgtcactcggggatcaggctagcatttcttgccgctcatctcagtctctggtccaccggaatgggaacacatacctccattggtacctccagaaacctggacagagccctaaactgctcatccacaaagtctcaaatcggttctccggcgtgcccgatcgctttagcggatccggatctgggaccgacttcacactgaaaatctcacgagtggaggctgaggatctcggcgtctacttctgtagtcagagtacccacgtcccacccctcacctttggcgctggaacaaaactggagctgaaacgagccgatgctgctcctaccgtgtccatctttcctggctccgggggaggcgggagcggaggcgaagtgaaactccagcagtctggcccttctctcgtggaacctggcgcttctgtgatgatctcctgtaaggcctctggatcttcctttaccggctacaacatgaactgggtccggcagaacattggcaaatccctggaatggattggcgccatcgatccttactacggcggcacatcatacaatcagaaattcaaggggcgagcaacactcactgtcgacaaatcttcatccaccgcctacatgcacctgaaatctctcacatccgaggatagtgctgtctactactgtgtctctggcatggaatactggggacagggaacttctgtcaccgtgtctagtgccaaaaccacacctccctccgtgtacggacgagtcactgtctcatctgctgaaccaaaatcctgtgacaaaacacacacatgcccaccttgtcctgcccctgaactgctcggcggaccttccgtctttctgtttccccccaaacccaaggatacactcatgatttctaggacccccgaagtcacttgtgtcgtggtcgatgtgtctcacgaggatcctgaagtgaaattcaactggtacgtggacggagtcgaggtccacaatgccaaaacaaaaccccgggaggaacagtacaatagcacctaccgagtcgtgtccgtgctcaccgtcctccatcaggattggctgaacggcaaagagtacaagtgtaaagtgagtaacaaggctctccccgctcctattgaaaaaaccatctcaaaagcaaaaggccagcctagggagcctcaggtctacacactgccaccctcacgggacgaactcaccaaaaatcaggtgtccctcacttgcctggtgaaaggcttctacccttccgatatcgctgtggaatgggagtcaaatgggcagcccgaaaacaactacaaaacaaccccccctgtgctcgattccgatggctcttttttcctgtactccaaactcaccgtggacaaatcacgctggcagcaggggaatgtcttttcttgctccgtgatgcacgaggccctccacaatcattacacccagaaatccctctcactctcacccggcaaaaaggaccctaaaaccacgacgccagcaccgcgaccaccaacaccggcgccaaccatcgcatcgcagcccctgtccctgcgcccagaggcgtgccgaccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggcagcggcgccaccaacttcagcctgctgaagcaggccggcgacgtggaggaaaaccctggccccatgtctttaagaggctctttatctcgtctgctgcagactcgtgtgcacagcattttaaaaaagagcgtgcacagcgtggccgtcattggagcccccttcagccaaggccagaagagaaagggcgtcgaacacggacccgccgccatcagagaagctggtttaatgaagagactgagctctttaggctgccatttaaaagacttcggagatttgtcttttacccccgtccccaaggacgatttatacaataatttaatcgtgaaccccagatccgtgggactggctaaccaagaactggccgaggtcgtgagcagagccgtgtccgacggctactcttgtgtgactttaggcggcgatcactctttagccattggcacaatctccggacacgctaggcactgccccgatttatgcgtggtgtgggtggacgctcacgccgacatcaatacccctctgaccaccagcagcggcaatttacacggacagcccgtcagctttttactgagggagctgcaagataaggtgcctcagctgcccggcttcagctggatcaagccttgtatcagcagcgcttccatcgtgtacattggtttaagagacgtggaccctcccgaacacttcatcctcaagaactacgacattcagtacttcagcatgagggatatcgatcgtctcggaatccagaaggtgatggaaaggaccttcgatttactcatcggaaagaggcagaggcctatccatttatccttcgacatcgacgccttcgatcctacactggcccccgctactggtacacccgttgtgggcggtttaacctatagggagggcatgtacatcgccgaagagatccacaacaccggtttactgagcgctctggatttagtggaggtgaatcctcagctggccacctccgaggaggaggccaaaaccaccgccaatctggccgtggacgtgatcgccagctccttcggccagaccagagagggcggccacattgtgtacgaccagctgcccacacccagctcccccgatgagtccgaaaaccaagctcgtgtcagaatctgataataacccaagcttaacacgagccatagatagaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactgcccacctcgggggtctttcatt

(19) United States
(12) Patent Application Publication
(10) Pub. No.: US 2022/0378828 A1
(43) Pub. Date: Dec. 1, 2022

(54) FUSION PROTEINS WITH ARGINASE ACTIVITY

(71) Applicant: CANCER RESEARCH TECHNOLOGY LIMITED, London (GB)

(72) Inventors: Francis Jay Mussai, Birmingham West Midlands (GB); Carmela De Santo, Birmingham West Midlands (GB)

(73) Assignee: CANCER RESEARCH TECHNOLOGY LIMITED, London (GB)

(21) Appl. No.: 17/621,182

(22) PCT Filed: Jun. 29, 2020
(86) PCT No.: PCT/GB2020/051571

§ 371 Date: Dec. 20, 2021

(30) Foreign Application Priority Data
Jun. 27, 2019 (GB) 1909283.2

(51) Int. Cl. A61K 35/17; C07K 14/725; C07K 14/705; C07K 16/30; C07K 16/28; C07K 16/18; C12N 9/78

(52) U.S. Cl. CPC A61K 35/17; C07K 14/7051; C07K 14/70521; C07K 14/70578; C07K 16/3084; C07K 16/2803; C07K 16/18; C07K 16/2863; C12N 9/78; C07K 2319/00; A61K 38/00

(57) ABSTRACT

The invention relates to fusion target-binding proteins, such as chimeric antigen receptors (CARs), that comprise a target binding moiety, an intracellular signalling region, and an arginase domain. These proteins confer advantages that include improved cell killing and increased proliferation. The invention also relates to nucleic acids encoding the fusion target-binding proteins and cells expressing such proteins. The invention relates to pharmaceutical compositions, medical uses, and methods of treatment, all using the fusion target-binding proteins, cells, or nucleic acids disclosed. The medical uses and methods of treatment are of particular benefit in cancer therapy.

### CROSS-REFERENCE TO RELATED APPLICATIONS

This application is a National Phase of International Application No. PCT/GB2020/051571 filed Jun. 29, 2020, which designated the U.S. and that International Application was published under PCT Article 21(2) in English. This application also includes a claim of priority under 35 U.S.C. § 119(a) and § 365(b) to British Application No. GB 1909283.2 filed Jun. 27, 2019, the entirety of which is hereby incorporated by reference.

### REFERENCE TO A SEQUENCE LISTING

This application containing an ASCII text file, with a sequence listing, named “SequenceListing065493_000022us00_ST25”, created on Dec. 17, 2021, and having 72 kilobytes in size is in incorporated herein by reference as though fully set forth in its entirety.

### FIELD OF THE INVENTION

The present invention relates to fusion target-binding proteins, and to cells comprising such proteins. It also relates to nucleic acids encoding fusion target-binding proteins. The invention relates to pharmaceutical compositions, medical uses, and methods of treatment, all using the fusion target-binding proteins, cells, or nucleic acids disclosed.

### BACKGROUND

Fusion proteins with target-binding capabilities have been used in a number of therapeutic applications. Most notably, T cells engineered to express chimeric antigen receptors (CARs) have been used in the treatment of cancer. However, as discussed further below, despite showing considerable clinical promise, such treatments have not been universally effective.

CAR-T Failure in Pre-Clinical and Clinical Studies

Despite advances in cytotoxic chemotherapy for both adult and paediatric cancers, it is clear that a number of major cancer subtypes still have an extremely poor prognosis. Immune therapies provide an alternative approach to targeting the malignant cancer cells directly, and avoid the toxic side-effects to normal cells of standard approaches.

Chimeric Antigen Receptor (CAR)-T cells (CAR-T) are autologous patient-derived T cells which have been engineered, typically with an antibody fragment (scFv), to specifically recognise surface antigens on tumour cells. The proof-of-principle of using CAR-T cells to successfully treat paediatric cancers has been established in patients with chemo-resistant, relapsed paediatric B Acute Lymphoblastic Leukaemia who underwent rapid and sustained remissions using anti-CD19 CAR T cells. In solid tumours neuroblastoma, the most common solid cancer of childhood, has been the model of choice and proved highly informative in the response of solid tumours to CAR-T cell therapy. Preclinical studies indicate that CAR T cells that recognise disialoganglioside 2 (GD2) antigen could represent a powerful new way of killing neuroblastoma cells. Although neuroblastoma has become the paradigm for CAR-T cell development against solid tumours, only limited anti-tumour efficacy has been seen in preclinical models and early phase trials. First generation anti-GD2 CAR T cells failed to persist in vivo and had minimal anti-tumour effects. Second generation anti-GD2-CAR T cells (with CD28 or 4-1BB costimulatory domains) had improved persistence in vivo, leading to moderate tumour regressions, but become functionally exhausted in the presence of neuroblastoma. In humans, a study of anti-GD2 CAR T cells made the key observation that despite infusion of large numbers of these cells, CAR T cell numbers become low or undetectable within weeks, and that the majority of patients with active disease did not achieve a complete remission. Importantly patients who had low-level persistence of CAR T cells had a longer survival. These findings suggest that the local and systemic tumour microenvironment impairs persistence of CAR-T cells, despite the presence of large target antigenic load on residual neuroblastoma tumours.

CAR-T cell therapy has also been tested against a limited number of other solid tumours in vitro, in vivo, and in man. In each case results against these malignancies have failed to replicate the exciting data found for anti-CD19 CAR-T cells in ALL.

Acute Myeloid Leukaemia

Acute Myeloid Leukaemia, is the most common acute leukaemia of adults and the second most common leukaemia of childhood. Incidence increases with age, and for patients with high risk or relapsed disease the prognosis is extremely poor with survival <12 months in adults, despite haematopoietic stem cell transplant. For elderly patients or those with co-existing morbidities standard chemotherapeutic regimens are poorly tolerated leading to sub-optimal treatment, and an in ability to achieve cure. Few effective new drugs have been developed for AML, as such immunotherapeutics offers the potential of a different approach. CD33 is almost universally expressed on AML blasts and has proved to be an effective target for immunotoxin-based therapeutics (Gemtuzumab ozogamicin). Anti-CD33 CAR-T cells are cytotoxic to AML blasts in vitro and eradicate leukaemic burden in vivo. On this basis a Phase I clinical trial of anti-CD33 CAR T cells has been initiated in China (NCT01864902 and NCT02958397). Reports from 1 patient with chemo-refractory AML showed a reduction in bone marrow AML blasts. These results provide proof-of principle that anti-CD33 CAR T cells can be effective. However disease relapsed by 9 weeks post CAR infusion despite measurable CAR-T cells remaining in both the blood and bone marrow. The finding suggests that the CAR-T cells have been rendered inactive, by the leukaemia microenvironment (no evidence for CD33 loss on AML blasts as a mechanism of escape).

Mesothelioma, Ovarian and Pancreatic Cancer

Mesothelioma, an asbestos related tumour with almost universally poor prognosis in adults, expresses the cell surface glycoprotein mesothelin. Mesothelin is also expressed on epithelial cancers, such as ovarian, lung adenocarcinoma, and pancreatic cancer. Mesothelin has been demonstrated to be an effective and selective target for passive immunotherapy with immunotoxins such as SS1P leading to its choice for development in CAR T technologies. In murine models anti-mesothelin CAR-T cells demonstrate clear and persistent anti-tumour activity. Anti-mesothelin CAR-T cells have also been administered to patients with these tumours and although limited responses were detected (PR, SD) in each case the tumours progressed. CAR-T cell persistence was extremely poor with cells becoming undetectable within only days of initial or repeat administration. Even when CAR-T cells are placed within the tumour, and hence in close proximity to target antigen, responses remain muted suggesting a strong immunosuppressive microenvironment that reduces the function of the T cells.

Glioblastoma

Glioblastoma is one of the most devastating brain tumours of both adults and children, with patients frequently experiencing a rapid disease progression and treatment failure despite intensive chemotherapy and radiotherapy based regimens. Glioblastomas express a variant of the Epidermal Growth Factor Receptor—EGFRvIII, providing a tumour-specific antigen which can be targeted by immunotherapy. EGFRvIII may also be expressed on approximately one third of advanced colorectal cancers. Anti-EGFRvIIII CAR-T cells demonstrated disease control of glioblastomas in orthotopic murine xenografts. However in all cases tumours continue to grow, leading to murine death, despite detectable levels of CAR-T cells in all organs including the brain. Again this data suggests that the CAR-T cells are inactivated by the tumour microenvironment. A Phase I trial based on this rationale is currently underway (NCT02844062, NCT02664363).

Arginine and the Immunosuppressive Microenvironment

Arginine is a semi-essential amino acid, required by healthy tissues for a number of cell processes including cell viability, proliferation and protein synthesis. Whole body arginine levels are maintained principally through dietary intake, and to a lesser extent by synthesis from precursors in an ‘intestinal-renal axis’. At a cellular level, arginine is imported from the extracellular fluid via Cationic Amino Acid (CAT; SLC7A) family of transporters and enters the urea cycle. In conditions of high demand such as inflammation, pregnancy, and cancer, arginine levels can become limited in the local tissue microenvironment and systemically. Some tissues and cells can protect themselves by resynthesizing arginine from precursors, through the expression of ArgininoSuccinate Synthase (ASS1) and Ornithine Transcarbamylase (OTC). Cells which lack expression of at least one of these enzymes are dependent on import of extracellular arginine, a state known as arginine auxotrophism.

Following transport across the cell membrane arginine is metabolised principally by the enzymes Arginase 1 (ARGI), Arginase II (ARGII), and nitric oxide synthase (NOS). Arginase 1 and Arginase 2 catabolise the conversion of arginine into urea and ornithine. Although similar in function, these 2 enzyme isoforms have notable differences. Arginase I is encoded on Chromosome 6, has a cytoplasmic localisation, and is predominantly expressed in hepatocytes. Arginase 1 knock-out in murine model is a lethal phenotype, and congenital arginase 1 deficiency in humans leads to a profound, progressive neuro- and metabolic-deficiency which is life-threatening and -limiting. Arginase II is encoded on Chromosome 14, and shares around a 60% homology to Arginase 1. Arginase II is mostly located inside cell mitochondria, although cytoplasmic localisation may occur in the pathological setting. It is more widely expressed in a number of cell types, and tissues, and unlike Arginase I murine knock-outs have minimal physiological consequences, and no human phenotype has been identified. Production of ornithine by both arginase isoforms feeds forwards to drive a number of different cell processes essential for cell proliferation and survival. For nitric oxide synthase, this enzyme mediates the conversion of arginine into nitric oxide species for cell signalling and control of infectious pathogens.

Previous studies have suggested that inhibition of arginase at the tumour site may be beneficial in addressing the issues of poor CAR T cell activity in vivo. Patient-derived, and engineered T cells are highly dependent on the uptake and metabolism of arginine for cell proliferation and activity, as they are unable to re-synthesise their own arginine adequately. As a result depletion of local and systemic arginine by tumour cells or circulating/infiltrating myeloid cells or artificial conditions in the laboratory or clinical setting leads to a significant impairment and failure in T cell expansion and function. Inhibition of arginine metabolism in tumour cells or circulating/infilitrating myeloid cells, through non-clinical small molecule inhibitors, leads to restoration of autologous and engineered T cell (for example CAR-T, antigen-specific T cells, iNKT, NK cell) activation, proliferation and cytotoxicity. However clinical strategies to inhibit arginine metabolism in cancer patients, have been limited due to the failure of such drugs to adequately inhibit tumour and myeloid cell arginase activity in patients and drug toxicities.

### SUMMARY OF THE INVENTION

According to a first aspect of the invention there is provided a fusion target-binding protein comprising a target binding moiety, an intracellular signalling region, and an arginase domain. For the sake of brevity, these may be referred to as “proteins of the invention” herein. By way of non-limiting example, a fusion target-binding protein of the invention may be a chimeric antigen receptor (CAR).

According to a second aspect of the invention there is provided a cell comprising a fusion target-binding protein comprising a target binding moiety, an intracellular signalling region, and an arginase domain. Such cells are also referred to as “cells of the invention” herein. As discussed further below, a cell of the invention may be a leukocyte, and particularly a T cell. A cell of the invention may express a protein of the invention.

According to a third aspect of the invention, there is provided a nucleic acid molecule encoding a fusion target-binding protein comprising a target binding moiety, an intracellular signalling region, and an arginase domain. These may be referred to herein as “nucleic acid molecules of the invention”.

According to a fourth aspect of the invention there is provided a pharmaceutical composition comprising a protein of the first aspect of the invention, a cell of the second aspect of the invention, or a nucleic acid molecule of the third aspect of the invention and a pharmaceutically acceptable carrier or diluent.

According to a fifth aspect of the invention, there is provided a protein of the first aspect of the invention, a cell of the second aspect of the invention, a nucleic acid molecule of the third aspect of the invention, or a pharmaceutical composition of the fourth aspect of the invention for use in the prevention and/or treatment of a disease. The disease may be cancer. A suitable cancer may be selected from the group consisting of: neuroblastoma; acute myeloid leukaemia (AML); mesothelioma; ovarian cancer; pancreatic cancer; and glioblastoma. Specific embodiments suitable for use in the prevention and/or treatment of these cancers are discussed further below.

According to a sixth aspect of the invention, there is provided a method of manufacturing a cell of the second aspect of the invention, the method comprising providing a cell with a nucleic acid molecule of the third aspect of the invention, such that the nucleic acid molecule is expressed by the cell to produce a fusion target-binding protein comprising a target binding moiety, an intracellular signalling region, and an arginase domain. Suitably the cell may be a T cell.

According to a seventh aspect of the invention, there is provided a method of preventing and/or treating a disease, the method comprising providing a protein of the first aspect of the invention, a cell of the second aspect of the invention, or a nucleic acid molecule of the third aspect of the invention, to a subject in need of such prevention and/or treatment. The disease may be cancer. As before, a suitable cancer may be selected from the group consisting of: neuroblastoma; acute myeloid leukaemia (AML); mesothelioma; ovarian cancer; pancreatic cancer; and glioblastoma.

According to an eighth aspect of the invention, there is provided a method of increasing proliferation of a leukocyte, the method comprising stimulating arginase activity within the leukocyte.

According to a ninth aspect of the invention, there is provided a method of increasing cytocidal activity of a leukocyte, the method comprising stimulating arginase activity within the leukocyte

The eighth and ninth aspects of the invention are based upon the inventors' findings that increased arginase activity within leukocytes increases both leukocyte proliferation and cytocidal activity. The leukocyte may be modified for therapeutic use. The leukocyte may be located in the blood. The leukocyte may be located in a tumour. The increase in arginase activity may optionally brought about by the expression of an exogenous arginase domain within the leukocyte. Such an exogenous arginase domain may be expressed as part of a fusion protein comprising the arginase domain.

According to a tenth aspect, the invention provides a leukocyte comprising an exogenous arginase domain. The exogenous arginase domain may be part of a fusion protein comprising this domain. The fusion protein may be a therapeutic fusion protein comprising an exogenous arginase domain. A suitable therapeutic fusion protein may be selected from the group consisting of: a chimeric antigen receptor comprising exogenous arginase domain; and a T cell receptor comprising an exogenous arginase domain.

Suitably a leukocyte in the eighth, ninth or tenth aspect of the invention may be a T cell. Suitable T cells are as considered elsewhere in the specification.

It will be appreciated that the various embodiments described on the following pages with reference to the proteins of the first aspect of the invention are also applicable to proteins referred to in other aspects of the invention (for example expressed by cells of the second aspect of the invention, encoded by nucleic acids of the third aspect of the invention, or incorporated in pharmaceutical compositions of the fourth aspect of the invention), except for where the context requires otherwise. Similarly, embodiments relating to arginase domains, cell types, therapeutic applications, and pharmaceutical compositions, or considerations relating to modified biological properties, in connection with the proteins of the first aspect of the invention or cells of the second aspect of the invention should, except for where the context requires otherwise, be taken as applicable to the methods of the eighth or ninth aspects of the invention, or the cells of the tenth aspect of the invention.

### BRIEF DESCRIPTION OF THE DRAWINGS

FIG. 1. Panels A and B show protein-enzyme constructs can be transduced into human T cells and Jurkat lines, assessed by measuring expression of tCD34 using flow cytometry. Panel A illustrates representative flow cytometry staining, and panel B sets out a summary of transduction efficiency across multiple T cell donors).

FIG. 2 illustrates the ability of the arginase domains present in fusion target-binding proteins of the invention expressed by transduced cells to perform their arginase function. The catabolism of arginine into ornithine and urea, by arginase type I or arginase type II domains, was assessed and compared to control constructs, without enzyme domains (GD2 only).

FIG. 3 illustrates specific cell lysis by T cells of the invention expressing fusion target-binding proteins comprising an arginase type I domain (GD2-ARG1), or an arginase type II domain (GD2-ARG2) on neuroblastoma cell line assessed against the control (GD2 only) under conditions of i) standard culture conditions, ii) arginine-free conditions, iii) supplemented arginine conditions.

FIG. 4 shows T cells of the invention comprising fusion target-binding proteins comprising arginase type I or arginase type II domains showed enhanced proliferation compared to the control (GD2 only) in standard (R10%), arginine Free (ARG−) and arginine supplemented (ARG+) conditions.

FIG. 5 confirms that arginase type I and arginase type II domains present in proteins of the invention demonstrate enzyme activity in transformed Jurkat T cells.

FIG. 6 shows the arginase type I and II domains in proteins of the invention demonstrate activity in human T cells.

FIG. 7 shows that arginase I and II enzyme domains in proteins of the invention enhance T cell proliferation in low arginine and tumour conditions, and do not adversely affect T cell exhaustion.

FIG. 8 shows that cells expressing proteins of the invention comprising arginase I and II enzyme domains have enhanced antigen-specific cytotoxicity against target tumour cells.

FIG. 9 shows that the presence of arginase enzyme domains in proteins of the invention leads to altered intracellular metabolic profiles.

FIG. 10 shows that presence of arginase enzyme domains in proteins of the invention can lead to altered mitochondrial respiration.

### DETAILED DESCRIPTION OF THE INVENTION

The present invention is based upon the inventors' finding that cells expressing fusion target-binding proteins comprising an arginase domain have increased ability to kill cancer cells as compared to prior art CAR-T cells, and that this is particularly notable in conditions representative of those found in tumours. This is highly advantageous, as immunosuppressive effects of the tumour microenvironment have contributed to the failures of many earlier CAR-based therapies.

In addition, cells of the invention exhibit an improved ability to proliferate as compared to control CAR-T cells, particularly in conditions representative of those found in the blood.

Cells of the invention, whether comprising an arginase type I or arginase type II domain, have demonstrated increased cytocidal killing of cancer cells, as compared to control CAR-T cells, under all conditions tested. These include “standard” concentrations of arginine, as well as conditions in which arginine concentrations are experimentally reduced (representative of conditions found in the tumour microenvironment) or increased.

The ability to kill target cells more effectively in reduced arginine conditions is likely to be beneficial in solid tumours, where lack of arginine is known to contribute to the immunosuppressive microenvironment. The use of proteins or cells of the invention, comprising arginase domains, in such applications is in contrast to suggestions in the prior art that therapeutic cells may be modified to overcome the immunosuppressive tumour microenvironment by decreasing local arginase activity.

The demonstration of increased cytocidal capacity in conditions where arginine is present at standard physiologically relevant levels, or at increased levels, indicates that the therapeutic utility of proteins and cells of the invention will go beyond use in the prevention and/or treatment of solid tumours. Cells in the blood are not exposed to conditions of reduced arginine concentration. Accordingly, proteins or cells of the invention utilising suitable target binding moieties may be used in the targeted killing of blood borne cells, such as blood borne cancer cells, or infected cells in the circulation.

Furthermore, the increased proliferative capacity of cells of the invention (whether comprising arginase type I or arginase type II domains) in conditions representative of the blood indicates that these cells may be able to expand their numbers in the circulation more effectively than can prior art CAR-T cells, and that this will provide expanded cell populations able to kill blood borne targets, or to migrate into solid tumours and kill cancer cells therein.

Accordingly, it will be recognised that the proteins and cells of the invention provide improved therapeutic agents as compared to CAR-based therapies of the prior art. The various aspects and embodiments of the invention described herein arise from, or contribute to, these improvements.

For the purposes of understanding the invention, it will now be further described with reference to the following definitions. For the sake of brevity the paragraphs that follow may refer to particular embodiments only in the context of proteins of the invention, but it will be appreciated that, except for where the context requires otherwise, embodiments referred to in connection with proteins of the invention may be employed in any of the other aspects of the invention disclosed herein.

Fusion Target-Binding Proteins

Fusion target-binding proteins are artificial fusion proteins that enable a desired specificity to be conferred on desired biological properties of a cell by which the protein of the invention is expressed. For the sake of brevity, they will also be referred to as “proteins” or “proteins of the invention” in the present disclosure. Different types of cells, and the desired biological properties that they are respectively able to provide in the context of the present invention, are discussed further elsewhere in the specification. Typically, in the context of medical uses of fusion target-binding proteins and cells expressing such proteins, cytocidal activity targeted against cells associated with a disease (such as cancer cells or infected cells) confers the required therapeutic utility.

It will be appreciated that it can readily be determined whether or not a protein (such as the fusion target-binding proteins of the invention) is artificial by comparing the amino acid sequence of the protein with a suitable database of naturally occurring proteins. The skilled person will be aware of a very large number of databases suitable for such use.

Proteins of the invention comprise at least a target binding moiety, an intracellular signalling region and an arginase domain. These terms are defined elsewhere within the present specification. The skilled person will appreciate that such proteins may also incorporate various other optional domains or regions.

The different portions of the fusion target-binding protein (target binding moieties, intracellular signalling regions, and arginase domains) may be derived from two or more different “sources”. Thus, the different portions may be derived from two or more naturally occurring molecules, such as proteins. Additionally, the different portions may be derived from different sources in terms of different originating kingdoms or species.

A class of fusion target-binding proteins of particular interest in the context of the present invention are chimeric antigen receptor (CAR) proteins. CARs utilise antibodies, or fragments thereof, to confer specificity of binding, and intracellular signalling regions to determine the specific biological activity required. Various different generations of CARs are known, and each of these different generations represents a suitable example of a fusion target-binding protein of the invention, unless the context of the present disclosure requires otherwise.

For the avoidance of doubt, proteins of the invention may also be taken as encompassing T cell receptors (TCRs) modified to comprise an arginase domain. In such embodiments, the target binding moiety may be provided by the TCR α and TCR β chains of the receptor. Since the target binding moiety and arginase domain are from different protein sources, such modified TCRs are fusion proteins for the purposes of the present invention.

Proteins of the invention typically further comprise additional portions, including one or more from the group consisting of: a transmembrane portion, a CH2CH3 spacer portion, a CD8 hinge portion, and a CD8a signalling portion.

The amino acid sequences of exemplary proteins of the invention are set out in SEQ ID NOs: 17 and 19. It will be appreciated that a molecule comprising or consisting of any of these sequences represents a protein in accordance with the first aspect of the invention. Any of the proteins set out in SEQ ID NOs: 17 and 19 may be utilised in the medical uses, methods of treatment, or pharmaceutical compositions of the invention.

Fragments or Variants of the Sequences in the Context of the Present Invention

The specification contains a number of exemplary protein and nucleic acid sequences. As well as the fusion target-binding proteins and nucleic acids encoding them, these include sequences of arginase domains, of antigen binding domains, and of intracellular signalling regions.

It will be appreciated that, except for where the context requires otherwise, the scope of the invention should not be limited to the specific exemplary sequences set out herein. In particular, the skilled reader will recognise that fragments or variants of the exemplary sequences may still be able to provide the required activity conferred by the exemplary sequences. Such suitable fragments or variants of the exemplary sequences may be utilised in the various aspects and embodiments of the invention.

In the context of the present invention, a fragment of a sequence should be taken as being a truncated version of the original sequence (i.e. not the full length sequence), but as sharing full sequence identity with a corresponding portion of the original sequence.

In contrast a variant of a sequence should be taken as being a protein or nucleic acid that shares a certain degree of identity with the original sequence (or with a particular fragment of the original sequence) but that includes at least one modification (for example, a substitution, addition, or deletion) as compared to the original sequence.

Accordingly, references in the present specification to exemplary amino acid or nucleic acid sequences should, except for where the context requires otherwise, be taken as also encompassing functional fragments or variants of the exemplary sequences. For example, a suitable fragment may comprise at least 60%, at least 65%, at least 70%, at least 75%, at least 80%, at least 85%, at least 90%, or at least 95% of the full length of a relevant exemplary sequence. Indeed, a suitable variant may comprise at least 96%, at least 97%, at least 98%, or at least 99% of the full length of the exemplary sequence.

A suitable variant may share at least 60%, at least 65%, at least 70%, at least 75%, at least 80%, at least 85%, at least 90%, or at least 95% identity with a relevant exemplary sequence. Indeed, a suitable variant may share at least 96%, at least 97%, at least 98%, or at least 99% identity with the relevant exemplary sequence.

That a fragment or variant is “functional” may be assessed experimentally, with reference to assays known to those skilled in the art, including those assays described in the Examples. In the case of components of the fusion target-binding proteins of the invention, such as target binding moieties or arginase domains, function may be determined with reference to the ability to bind a desired target, or to promote arginine catabolism. In the case of proteins of the invention, function may be determined with reference to biological activities such as cytocidal activity, or proliferation.

An Arginase Domain

The fusion target-binding proteins, such as CARs, of the invention comprise an arginase domain that catalyses the conversion of arginine and water to ornithine and urea. The ability of a domain to fulfil this function, that is to say to demonstrate arginase activity by promoting catabolism of arginine to ornithine and urea, may be investigated by any suitable means or assay.

Merely by way of example, suitable assays by which the ability of a domain to promote the catabolism of arginine may be investigated are described further in the Examples, in relation to the characterisation of exemplary cells of the invention. It will be appreciated that these assays may be used to qualitively or quantitively assess the ability of a domain of interest, such as a fragment or variant of a naturally occurring arginase domain, to function as an arginase domain suitable for use in the context of the invention. These assays may also be used in determining the proportion of arginase activity that a domain of interest exhibits as compared to a wild type arginase domain.

As referred to above, two isoforms of arginase are known, arginase type I and arginase type II. The amino acid sequence of human wild type arginase type I is set out in SEQ ID NO:1, and the amino acid sequence of human wild type arginase type II is set out in SEQ ID NO:2. DNA encoding human wild type arginase type I is set out in SEQ ID NO:3, and DNA encoding human wild type arginase type II is set out in SEQ ID NO:4.

An arginase domain may comprise all of a naturally occurring mammalian, and preferably human, arginase enzyme, or a fragment of such an enzyme. Alternatively, an arginase domain may comprise or variant of a naturally occurring mammalian, and preferably human, arginase enzyme or a variant of a fragment of such an enzyme.

Merely by way of example, a suitable arginase domain may comprise an arginase type I enzyme, or a fragment or derivative thereof.

Alternatively, a suitable arginase domain may comprise an arginase type II enzyme, or a fragment or derivative thereof.

As shown in the Examples section, the inventors have found that proteins of the invention comprising arginase type I or arginase type II as arginase domains both achieve comparable levels of transduction, and also comparable levels of arginase activity when expressed by cells of the invention.

A suitable arginase domain for use in accordance with the invention may achieve at least 50% of the arginase activity of human wild type arginase type I or arginase type II. Alternatively, a suitable arginase domain for use in accordance with the invention may achieve at least 55%, at least 60%, at least 65%, at least 70%, at least 75%, at least 80%, at least 85%, at least 90%, or at least 95% of the arginase activity of human wild type arginase type I or arginase type II. Indeed, suitable arginase domain for use in accordance with the invention may achieve 100% of the arginase activity of human wild type arginase type I or arginase type II.

A suitable fragment of the arginase type I enzyme set out in SEQ ID NO: 1 may comprise all but 1 of the amino acid residues set out in SEQ ID NO: 1, all but 2 of the amino acid residues set out in SEQ ID NO: 1, all but 3 of the amino acid residues set out in SEQ ID NO: 1, all but 4 of the amino acid residues set out in SEQ ID NO: 1, all but 5 of the amino acid residues set out in SEQ ID NO: 1, all but 6 of the amino acid residues set out in SEQ ID NO: 1, all but 7 of the amino acid residues set out in SEQ ID NO: 1, all but 8 of the amino acid residues set out in SEQ ID NO: 1, all but 9 of the amino acid residues set out in SEQ ID NO: 1, or all but 10 of the amino acid residues set out in SEQ ID NO: 1.

A suitable fragment of the arginase type I enzyme set out in SEQ ID NO: 1 may consist of up to 50%, up to 60%, up to 70%, up to 75%, up to 80%, up to 85%, up to 90%, or up to 95% of the sequence set out in SEQ ID NO: 1.

A suitable variant of the arginase type I enzyme set out in SEQ ID NO: 1 may share at least 75% identity with SEQ ID NO: 1, or with a fragment of SEQ ID NO: 1 as defined above. A suitable variant of may share at least 80% identity with SEQ ID NO: 1, or a fragment thereof; at least 85% identity with SEQ ID NO: 1, or a fragment thereof; at least 90% identity with SEQ ID NO: 1, or a fragment thereof; at least 95% identity with SEQ ID NO: 1, or a fragment thereof; at least 96% identity with SEQ ID NO: 1, or a fragment thereof; at least 97% identity with SEQ ID NO: 1, or a fragment thereof; at least 98% identity with SEQ ID NO: 1, or a fragment thereof; or at least 99% identity with SEQ ID NO: 1, or a fragment thereof.

A suitable fragment of the arginase type II enzyme set out in SEQ ID NO: 2 may comprise all but 1 of the amino acid residues set out in SEQ ID NO: 2, all but 2 of the amino acid residues set out in SEQ ID NO: 2, all but 3 of the amino acid residues set out in SEQ ID NO: 2, all but 4 of the amino acid residues set out in SEQ ID NO: 2, all but 5 of the amino acid residues set out in SEQ ID NO: 2, all but 6 of the amino acid residues set out in SEQ ID NO: 2, all but 7 of the amino acid residues set out in SEQ ID NO: 2, all but 8 of the amino acid residues set out in SEQ ID NO: 2, all but 9 of the amino acid residues set out in SEQ ID NO: 2, or all but 10 of the amino acid residues set out in SEQ ID NO: 2.

A suitable fragment of the arginase type II enzyme set out in SEQ ID NO: 2 may consist of up to 50%, up to 60%, up to 70%, up to 75%, up to 80%, up to 85%, up to 90%, or up to 95% of the sequence set out in SEQ ID NO: 2.

A suitable variant of the arginase type II enzyme set out in SEQ ID NO: 2 may share at least 75% identity with SEQ ID NO: 2, or with a fragment of SEQ ID NO: 2 as defined above. A suitable variant of may share at least 80% identity with SEQ ID NO: 2, or a fragment thereof; at least 85% identity with SEQ ID NO: 2, or a fragment thereof; at least 90% identity with SEQ ID NO: 2, or a fragment thereof; at least 95% identity with SEQ ID NO: 2, or a fragment thereof; at least 96% identity with SEQ ID NO: 2, or a fragment thereof; at least 97% identity with SEQ ID NO: 2, or a fragment thereof; at least 98% identity with SEQ ID NO: 2, or a fragment thereof; or at least 99% identity with SEQ ID NO: 2, or a fragment thereof.

In order to be suitable for use as arginase domains in the context of the invention, such fragments or variants should retain arginase activity as referred to above.

Target Binding Moieties

The proteins of the invention comprise a target binding moiety. Suitably the target binding moiety is an extracellular target binding moiety. Such moieties are particularly suitable for binding a target that is extracellular (with reference to the cell comprising the fusion target-binding protein).

The target binding moiety confers specificity of binding of the proteins, and hence of the cytocidal activity of the cells expressing proteins of the invention, to target structures, such as cells, on which a target molecule, recognised by the target binding moiety, is found.

In particular, the target binding moieties confer specificity of the biological activities of the cells of the invention (for example, cytocidal activity, or cell proliferation in response to activation) that underpin their therapeutic utility. Except for where the context requires otherwise, references to specific binding in the present disclosure may be interpreted as referring to a target binding moiety's ability to discriminate between possible partners in the environment in which binding is to occur. A target binding moiety that interacts with one particular target molecule when other potential targets are present is said to “bind specifically” to the target molecule with which it interacts. In some embodiments, specific binding is assessed by detecting or determining degree of association between the target binding moiety and its target molecule; in some embodiments, specific binding is assessed by detecting or determining degree of dissociation of a binding moiety-target molecule complex; in some embodiments, specific binding is assessed by detecting or determining ability of the target binding moiety to compete an alternative interaction between its target molecule and another entity. In some embodiments, specific binding is assessed by performing such detections or determinations across a range of concentrations. In a suitable embodiment, specific binding is assessed by determining the difference in binding affinity between cognate and non-cognate targets. For example, a target binding moiety that is specific may have a binding affinity for a cognate target molecule that is about 3-fold, 4-fold, 5-fold, 6-fold, 7-fold, 8-fold, 9-fold, 10-fold or more than binding affinity for a non-cognate target,

In the context of the present disclosure, “specificity” is a measure of the ability of a particular target binding moiety to distinguish its target molecule binding partner from other potential binding partners.

A suitable target binding moiety may be directed to any desired target molecule. The target binding moiety may be directed to a target molecule expressed exclusively, or extensively, by a target against which it is desired to direct the cytocidal activity of a cell expressing a protein of the invention. For example, a target binding moiety may be directed to a target molecule associated with a disease. Suitably the target binding moiety may be directed to a target molecule associated with cancer, or with an infection.

In a suitable embodiment, the target binding moiety is selected from the group consisting of: a GD2 target binding moiety; a CD33 target binding moiety; a mesothelin target binding moiety; an EGFRvIII target binding moiety; a VEGFR2 target binding moiety; a FAP target binding moiety; a EpCam target binding moiety; a GPC3 target binding moiety; a CD133 target binding moiety; a IL13Ra target binding moiety; a EphA2 target binding moiety; a Muc1 target binding moiety; a BCMA target binding moiety; a CD70 target binding moiety; a CD123 target binding moiety; a ROR1 target binding moiety; a PSMA target binding moiety; a CD5 target binding moiety; a GAP target binding moiety; a CEA target binding moiety; a PSCA target binding moiety; a Her2 target binding moiety; and a CD19 target binding moiety.

Examples of suitable GD2, CD33, mesothelin, and EGFRvIII target binding moieties are set out in SEQ ID NOs: 5 to 8 and 10. It will be appreciated that fragments or variants (for example, variants differing from the exemplary sequence by 1, 2, 3, 4, 5, or more amino acid residues) may be used as alternative target binding moieties, as long as the fragment or variant retains the ability to bind the target molecule.

Without limitation, suitable target binding moieties may be selected from the group consisting of: antibodies; antibody fragments (such as scFvs); derivatives of antibodies or their fragments; TCRs, such as TCR α chains or TCR β chains; and aptamers.

A GD2 Target Binding Moiety

A GD2 target binding moiety is a moiety capable of binding to disialoganglioside 2 (GD2), which may also be referred to as ganglioside GD2. A protein of the invention comprising a GD2 target binding moiety is suitable for use in circumstances in which it is desired to exert the cytocidal activity of a cell expressing a protein of the invention against a target comprising GD2 molecules, for example a cell expressing GD2.

GD2 is expressed by cancers of neuroectodermal origin, including neuroblastoma, osteosarcoma and melanoma. Therefore, it will be appreciated that a protein (such as a CAR) of the invention comprising a GD2 target binding moiety is suitable for use in circumstances in which it is desired to utilise a protein of the invention in a medical use for the prevention and/or treatment of any such GD2 expressing cancers, and particularly neuroblastoma.

A GD2 target binding moiety suitable for incorporation in a protein in accordance with the invention may be an anti-GD2 antibody, such as an anti-GD2 monoclonal antibody, or an antigen binding fragment or derivative thereof. For example, a GD2 target binding moiety may be an anti-GD2 scFv antibody fragment. Merely by way of example, a suitable GD2 targeting domain comprising an scFv antibody fragment is set out in SEQ ID NO: 5.

The scFv antibody fragment set out in SEQ ID NO: 5 is also referred to as the 14g2a scFv, as described in U.S. Pat. No. 9,493,740 B2. It is derived from the ch14.18 antibody disclosed in U.S. Pat. No. 9,777,068 B2, and it will be appreciated that other ch14.18 antibody fragments or variants may be used as GD2 target binding moieties in the proteins of the invention.

Alternatively, a suitable GD2 target binding moiety may be selected from the group consisting of: an anti-GD2 aptamer; or a fragment or derivative thereof.

Suitably a GD2 target binding moiety is capable of binding specifically to GD2.

A CD33 Target Binding Moiety

A CD33 target binding moiety is a moiety capable of binding to CD33 (also known as Siglec-3). CD33 is transmembrane protein. A protein of the invention comprising a CD33 target binding moiety is suitable for use in circumstances in which it is desired to exert the biological activity of a cell expressing a protein of the invention against a target comprising CD33 molecules, for example a cell expressing CD33.

CD33 is expressed by acute myeloid leukaemia (AML) cells. Therefore, it will be appreciated that a protein of the invention comprising a CD33 target binding moiety is suitable for use in circumstances in which it is desired to utilise a protein of the invention in a medical use for the prevention and/or treatment of a CD33 expressing cancer, such as AML.

A CD33 target binding moiety suitable for incorporation in a protein in accordance with the invention may be an anti-CD33 antibody, such as an anti-CD33 monoclonal antibody, or an antigen binding fragment or derivative thereof. For example, a CD33 target binding moiety may be an anti-CD33 scFv antibody fragment. Merely by way of example, a suitable CD33 targeting domain comprising an scFv antibody fragment is set out in SEQ ID NO: 6.

The scFv antibody fragment is set out in SEQ ID NO: 4 is derived from the humanised my96 clone monoclonal antibody. Details of the my96 antibody are set out in Leukemia. 2015 August; 29(8):1637-47, and details of the scFv fragment of SEQ ID NO: 6 are set out in US20160096892A1 (where this scFv is disclosed as SEQ ID NO: 147). It will be appreciated that other my96 antibody fragments or variants may be used as CD33 target binding moieties in the proteins of the invention.

Alternatively, a suitable CD33 target binding moiety may be selected from the group consisting of: an anti-CD33 aptamer; or a fragment or derivative thereof

Suitably a CD33 target binding moiety is capable of binding specifically to CD33.

A Mesothelin Target Binding Moiety

A mesothelin target binding moiety is a moiety capable of binding to mesothelin. Mesothelin is a 40 kDa protein that is the product of the MSLN. A protein of the invention comprising a mesothelin target binding moiety is suitable for use in circumstances in which it is desired to exert the biological activity of a cell expressing a protein of the invention against a target comprising mesothelin molecules, for example a cell expressing mesothelin.

Mesothelin is expressed by cells of a number of different types of cancers. Mesothelin expressing cancers include, for example, epithelial cancers, such as ovarian cancer, lung adenocarcinoma, and pancreatic cancer. Therefore, it will be appreciated that a protein of the invention comprising a mesothelin target binding moiety is suitable for use in circumstances in which it is desired to utilise a protein of the invention in a medical use for the prevention and/or treatment of any mesothelin expressing cancer.

A mesothelin target binding moiety suitable for incorporation in a protein in accordance with the invention may be an anti-mesothelin antibody, such as an anti-mesothelin monoclonal antibody, or an antigen binding fragment or derivative thereof. For example, a mesothelin target binding moiety may be an anti-mesothelin scFv antibody fragment. Merely by way of example, a suitable mesothelin targeting domain comprising an scFv antibody fragment is set out in SEQ ID NO: 7.

The scFv antibody fragment is set out in SEQ ID NO: 7 is derived from the SS1 antibody. Details of this antibody, and an scFV derived therefrom, are set out in WO 2015/090230 A (where the amino acid sequence of murine SS1 scFv is provided in SEQ ID NO: 279). It will be appreciated that other SS1 antibody fragments or variants may be used as mesothelin target binding moieties in the proteins of the invention.

Alternatively, a suitable mesothelin target binding moiety may be selected from the group consisting of: an anti-mesothelin aptamer; or a fragment or derivative thereof.

Suitably a GD2 target binding moiety is capable of binding specifically to GD2.

An EGFRvIII Target Binding Moiety

A EGFRvIII target binding moiety is a moiety capable of binding to epidermal growth factor receptor variant III (EGFRvIII). A protein of the invention comprising a EGFRvIII target binding moiety is suitable for use in circumstances in which it is desired to exert the biological activity of a cell expressing a protein of the invention against a target comprising EGFRvIII molecules, for example a cell expressing EGFRvIII.

EGFRvIII is expressed by a range of cancers of epithelial origin. Therefore, it will be appreciated that a protein of the invention comprising an EGFRvIII target binding moiety is suitable for use in circumstances in which it is desired to utilise a protein of the invention in a medical use for the prevention and/or treatment of cancers expressing EGFR, such as glioblastomas, and colorectal cancers. In particular, a protein of the invention comprising an EGFRvIII target binding moiety is suitable for use in the prevention and/or treatment of an EGFRvIII expressing cancer, such as glioblastoma.

An EGFRvIII target binding moiety suitable for incorporation in a protein in accordance with the invention may be an anti-EGFRvIII antibody, such as an anti-EGFRvIII monoclonal antibody, or an antigen binding fragment or derivative thereof. For example, a EGFRvIII target binding moiety may be an anti-EGFRvIII scFv antibody fragment. Merely by way of example, a suitable EGFRvIII targeting domain comprising an scFv antibody fragment is set out in SEQ ID NO: 8.

The scFv antibody fragment is set out in SEQ ID NO: 8 is derived from the 139 antibody disclosed in WO 2012/138475 A1 (in which a human scFV of the 139 antibody is set out as SEQ ID NO: 5, and a CAR construct incorporating the scFv is set out as SEQ ID NO: 11). It will be appreciated that other 139 antibody fragments or variants may be used as mesothelin target binding moieties in the proteins of the invention.

An alternative EGFRvIII target binding moiety may be derived from the MR1 anti-EGFRvIII antibody. An example of such an EGFRvIII target binding moiety is the scFv (derived from MR1) encoded by the DNA sequence of SEQ ID NO: 9. This amino acid sequence of this alternative EGFRvIII target binding moiety is set out in SEQ ID NO: 10.

Alternatively, a suitable EGFRvIII target binding moiety may be selected from the group consisting of: an anti-EGFRvIII aptamer; or a fragment or derivative thereof.

Suitably a EGFRvIII target binding moiety is capable of binding specifically to EGFRvIII.

Intracellular Signalling Regions

The proteins of the invention comprise at least one intracellular signalling region. The intracellular signalling region serves to couple binding of the target binding moiety to a target molecule with other biological activities of the cell expressing the protein. In particular, a suitable intracellular signalling region may couple binding of the target binding moiety to its target molecule with activation of the cell's cytocidal activity and/or to the cells ability to proliferate in response to activation.

As set out in the Examples, a suitable intracellular signalling region may activate cytotoxic or specific cytolytic activity in response to binding of the target molecule to the target binding moiety. Alternatively, or additionally, a suitable intracellular signalling region may facilitate activation-induced cell proliferation in response to binding of the target molecule to the target binding moiety.

In a suitable embodiment, the intracellular signalling region comprises a region selected from the group consisting of: a 4-1BB signalling region; an OX-40 signalling region; a CD28 signalling region; an ICOS signalling region; and a CD3 ζ signalling region.

It will be appreciated that proteins in accordance with the invention may comprise a plurality of intracellular signalling regions. Suitably the plurality may comprise more than one copy of an individual intracellular signalling region. For example, a protein of the invention may comprise multiple copies of one, or more, of: a 4-1BB signalling region; an OX-40 signalling region; a CD28 signalling region; an ICOS signalling region; and a CD3 ζ signalling region.

Additionally, or alternatively, a protein of the invention may comprise a combination of multiple intracellular signalling regions. For example, a protein in accordance with the invention may comprise a combination of intracellular signalling regions selected from the group consisting of: a 4-1BB signalling region; an OX-40 signalling region; a CD28 signalling region; an ICOS signalling region; and a CD3 ζ signalling region. Merely by way of example, a protein of the invention may comprise both a 4-1BB signalling region and a CD3 ζ signalling region.

A suitable a 4-1BB signalling region is one that is able to provide sufficient costimulatory signalling to a cell expressing a protein comprising such a signalling region to promote at least one of: activation of the cell, and/or function of the cell, such as cytokine release by the cell, and/or cytotoxicity by the cell; and/or proliferation and/or persistence of the cell. This persistence may be persistence of the in vivo or in vitro. The persistence may, in particular, be persistence of the cell in conditions of the immunosuppressive tumour microenvironment, or that replicate this microenvironment. By way of example, the cytokine release may include one or more cytokines from the group consisting of: IFN-gamma, and/or TNFα, and/or IL2.

Suitably the 4-1BB signalling region may comprise the full-length sequence of 4-1BB. Alternatively, a 4-1BB signalling region may comprise a truncated and/or modified form of the full-length sequence. Merely by way of example, a suitable 4-1BB signalling region may comprise the amino acid sequence set out in SEQ ID NO: 11, or a portion of this sequence. Suitably a 4-1BB signalling region for incorporation in a protein of the invention may consist of the amino acid sequence set out in SEQ ID NO: 11.

In a suitable embodiment, an OX-40 signalling region is one that is able to provide sufficient costimulatory signalling to a cell expressing a protein comprising such a signalling region to promote at least one of: activation of the cell, and/or function of the cell, and/or persistence of the cell. This persistence may be persistence of the in vivo or in vitro. The persistence may, in particular, be persistence of the cell in conditions of the immunosuppressive tumour microenvironment, or that replicate this microenvironment.

Suitably the OX-40 signalling region may comprise the full-length sequence of OX-40. Alternatively, an OX-40 signalling region may comprise a truncated and/or modified form of the full-length sequence. Merely by way of example, a suitable OX-40 signalling region may comprise the amino acid sequence set out in SEQ ID NO: 12, or a portion of this sequence. Suitably an 4-1 OX-40 BB signalling region for incorporation in a protein of the invention may consist of the amino acid sequence set out in SEQ ID NO: 12.

A suitable CD28 signalling region is one that is able to provide sufficient costimulatory signalling to a cell expressing a protein comprising such a signalling region to promote at least one of: activation of the cell, and/or function of the cell (and/or persistence of the cell. This persistence may be persistence of the in vivo or in vitro. The persistence may, in particular, be persistence of the cell in conditions of the immunosuppressive tumour microenvironment, or that replicate this microenvironment.

Suitably the CD28 signalling region may comprise the full-length sequence of CD28. Alternatively, a CD28 signalling region may comprise a truncated and/or modified form of the full-length sequence. Merely by way of example, a suitable CD28 signalling region may comprise the amino acid sequence set out in SEQ ID NO: 13, or a portion of this sequence. Suitably a CD28 signalling region for incorporation in a protein of the invention may consist of the amino acid sequence set out in SEQ ID NO: 13.

An ICOS signalling region is one that is able to provide sufficient costimulatory signalling to a cell expressing a protein comprising such a signalling region to promote at least one of: activation of the cell, and/or function of the cell, such as cytokine release by the cell, and/or cytotoxicity by the cell; and/or proliferation and/or persistence of the cell. This persistence may be persistence of the in vivo or in vitro. The persistence may, in particular, be persistence of the cell in conditions of the immunosuppressive tumour microenvironment, or that replicate this microenvironment.

Suitably the ICOS signalling region may comprise the full-length sequence of ICOS (also known as CD278). Alternatively, an ICOS signalling region may comprise a truncated and/or modified form of the full-length sequence. Merely by way of example, a suitable ICOS signalling region may comprise the amino acid sequence set out in SEQ ID NO: 14, or a portion of this sequence. Suitably an ICOS signalling region for incorporation in a protein of the invention may consist of the amino acid sequence set out in SEQ ID NO: 14. A truncated or modified form of ICOS may comprise at least the YMFM motif found at residues 180-183 of the full-length ICOS protein.

A suitable CD3 ζ signalling region is one that is able to activate a functional response within the T cell (e.g. cytokine release (e.g. interferon-gamma, TNFa and/or IL2), cytotoxicity and/or proliferation.)

Suitably the CD3 ζ signalling region may comprise the full-length sequence of CD3 ζ. Alternatively, a CD3 ζ signalling region may comprise a truncated and/or modified form of the full-length sequence. Merely by way of example, a suitable CD3 ζ signalling region may comprise the amino acid sequence set out in SEQ ID NO: 15 or SEQ ID NO: 16, or a portion of these sequences. Suitably a CD3 ζ signalling region for incorporation in a protein of the invention may consist of the amino acid sequences set out in SEQ ID NO: 15 or SEQ ID NO: 16.

Proteins of the Invention Targeting GD2

A protein of the invention that targets GD2 may comprise a GD2 targeting moiety in combination with a suitable intracellular signalling region (such as a 4-1BB intracellular signalling region and a CD3 ζ intracellular signalling region) and an arginase domain. The arginase domain may comprise or consist of arginase type I, arginase type II, or fragments or variants of these enzymes.

The inventors have found that cells expressing proteins of the invention comprising a GD2 targeting moiety in combination with either an arginase type I or arginase type II domain have particularly improved cytocidal activity, as compared to control CAR-T cells, in experimentally arginine-depleted conditions representative of the tumour microenvironment. This effect was most notable in cells of the invention comprising an arginase I domain, indicating a particular utility for these cells in the treatment of solid tumours.

The inventors have also found that cells expressing proteins of the invention comprising a GD2 targeting moiety in combination with an arginase type I or arginase type II domain have improved cytocidal activity, as compared to control anti-GD2 CAR-T cells, in conditions representative of the blood (“standard” or “arginine supplemented” conditions). This effect was particularly notable in respect of cells comprising an arginase II domain, indicating particular suitability of these cells in the treatment of blood-borne cancers.

These advantages are discussed further in the Examples section of the specification.

The amino acid sequence of exemplary proteins of the invention that target GD2 are set out in SEQ ID NOs: 17 and 19, which respectively incorporate arginase type I and arginase type II domains. The present invention should be taken as encompassing not only these specific proteins, but also as encompassing variants of these proteins that share the biological activity (particularly the enhanced cytocidal activity and increased proliferation) of this exemplary protein. Such variants may share at least 80% sequence identity with the protein of SEQ ID NO: 17 or 19. Suitably, such variants may share at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the protein of SEQ ID NO: 17 or 19.

Proteins of the Invention Targeting Mesothelin

A protein of the invention that targets mesothelin may comprise a mesothelin targeting moiety derived from the anti-mesothelin SS1 antibody, in combination with a suitable intracellular signalling region (such as a 4-1BB intracellular signalling region and a CD3 ζ intracellular signalling region) and an arginase domain. The arginase domain may comprise or consist of arginase type I, arginase type II, or fragments or variants of these enzymes.

Proteins of the Invention Targeting CD33

A protein of the invention that targets CD33 may comprise a CD33 targeting moiety derived from the anti-CD33 my96 antibody, in combination with a suitable intracellular signalling region (such as a 4-1BB intracellular signalling region and a CD3 ζ intracellular signalling region) and an arginase domain. The arginase domain may comprise or consist of arginase type I, arginase type II, or fragments or variants of these enzymes.

Proteins of the Invention Targeting EGFRvIII

A protein of the invention that targets EGFRvIII may comprise an EGFRvIII targeting moiety derived from the anti-EGFRvIII 139 antibody, in combination with a suitable intracellular signalling region (such as a 4-1BB intracellular signalling region and a CD3 ζ intracellular signalling region) and an arginase domain. The arginase domain may comprise or consist of arginase type I, arginase type II, or fragments or variants of these enzymes.

Nucleic Acids Encoding Proteins of the Invention

The third aspect of the invention provides a nucleic acid encoding a protein of the invention. The proteins may be in accordance with any of the aspects or embodiments of the invention described herein.

Suitably a nucleic acid in accordance with the invention comprises DNA. In a suitable embodiment, a nucleic acid of the invention comprises RNA. It will be appreciated that a suitable nucleic acid may essentially consist of DNA, may essentially consist of RNA, or may comprise a combination of DNA and RNA.

Examples of nucleic acids encoding proteins of the invention are set out in SEQ ID NOs: 18 and 20. These nucleic acid sequences are DNA molecules encoding exemplary proteins set out in the specification as follows:

| Nucleic acid of the invention | Encodes protein of the invention |
|---|---|
| set out in SEQ ID NO | set out in SEQ ID NO |
| 18 | 17 |
| 20 | 19 |

It will be appreciated that codon degeneracy means that there can be notable differences in the sequences of nucleic acids of the invention encoding a single given protein of the invention.

Merely by way of example, a suitable nucleic acid of the invention may share at least 70% sequence identity with one of the exemplary nucleic acids of the invention set out in SEQ ID NOs: 18 or 20. A suitable nucleic acid of the invention may share at least 75% sequence identity; at least 80% sequence identity; at least 85% sequence identity; at least 90% sequence identity; at least 95% sequence identity; at least 96% sequence identity; at least 97% sequence identity; at least 98% sequence identity; or even 99% or more sequence identity with one of the exemplary nucleic acids of the invention set out in SEQ ID NOs: 18 or 20.

A nucleic acid sequence encoding a protein of the invention that targets mesothelin may be the same as the nucleic acid sequences of any of SEQ ID NOs: 18 or 20 save that the part of those nucleic acid sequences that encodes the target binding moiety is replaced with a nucleic acid sequence that encodes SEQ ID NO: 7. A protein encoded by such a nucleic acid constitutes a protein of the invention.

A nucleic acid sequence encoding a protein of the invention that targets CD33 may be the same as the nucleic acid sequences of any of SEQ ID NOs: 18 or 20 save that the part of those nucleic acid sequences that encodes the target binding moiety is replaced with a nucleic acid sequence that encodes SEQ ID NO: 6. A protein encoded by such a nucleic acid constitutes a protein of the invention.

A nucleic acid sequence encoding a protein of the invention that targets EGFRvIII may be the same as the nucleic acid sequences of any of SEQ ID NOs: 18 or 20 save that the part of those nucleic acid sequence that encodes the target binding moiety is replaced with a nucleic acid sequence that encodes SEQ ID NO: 8 or 10. A protein encoded by such a nucleic acid constitutes a protein of the invention.

A nucleic acid encoding a protein of the invention may be provided in the form of a vector. Suitably the vector may be a viral vector, such as a retroviral vector or a lentiviral vector, or a transposon. Both retroviral and lentiviral approaches have been used successfully in the production of cells of the invention.

Cells of the Invention

The second aspect of the invention provides a cell comprising a protein in accordance with the first aspect of the invention. The cell may express the protein. The protein may be in accordance with any of the embodiments of the first aspect of the invention described herein.

Suitably a cell in accordance with the second aspect of the invention is a cell able to exert a cell-mediated immune response. A suitable cell may be able to exert cytocidal activity, for example by cytotoxic action, or by inducing specific cell lysis. Additionally, a suitable cell may be able to proliferate in response to binding of the protein to its corresponding target molecule. Suitably, a cell in accordance with the second aspect of the invention may be selected from the group consisting of: a T cell; and a natural killer (NK) cell.

Suitably a T cell may be selected from the group consisting of: an invariant natural killer T cell (iNKT); a natural killer T cell (NKT); a gamma delta T cell (gd T cell); an alpha beta T cell (ab T cell); an effector T cell; and a memory T cell.

Suitably a T cell may be selected from the group consisting of: a CD4⁺ lymphocyte; and a CD8⁺ lymphocyte.

The cell may be from a subject requiring prevention and/or treatment of a disease, such as cancer. The cell may be taken from a sample from such a subject.

Alternatively, the cell may be from a healthy donor subject (for the purposes of the present disclosure taken as a subject not afflicted with the disease to be treated with the protein or cell of the invention).

It will be appreciated that suitable cells may also include cells of cell lines.

Standard techniques for the collection of human cells, and their transformation with proteins such as the proteins of the invention, are well known to those skilled in the art. Preferred techniques for the retroviral transduction of human T cells, determination of transduction efficiency, and sorting of transduced T cells by magnetic activated cell sorting, are described further in the Examples.

Biological Activity of Cells of the Invention

Cells of the invention, comprising proteins of the invention exhibit a number of activities that are of benefit in applications such as the prevention and/or treatment of diseases.

These biological activities may be further considered with reference to cytocidal activities (which represent the means by which the cells of the invention are able to exert their therapeutic effects) and activities such as proliferation (for example in response to activation) which may contribute to increased persistence in vivo, thereby enabling the cells of the invention to exert their therapeutic effects for longer than has been the case for CAR-expressing cells of the prior art.

These respective biological activities are described further below. It will be appreciated that the advantages offered by the proteins and cells of the invention may arise as a result of the combination of these biological activities.

Biological activity of the cells of the invention may be determined with reference to suitable comparator cells. Examples of suitable comparator cells include cells of the same type as those of the invention that have not been transduced with a protein, or cells of the same type as those of the invention that have been transduced with a protein that does not comprise an arginase domain.

Cytocidal Activity of Cells of the Invention

For the purposes of the present invention, cytocidal activity should be taken as encompassing any activity by which cells of the invention (for example cells expressing proteins of the invention) kill other cells. By way of example, the killing of other cells may be achieved by means of cytotoxic action of the cells of the invention, or by specific cell lysis mediated by the cells of the invention.

The cells of the invention exert their cytocidal activity in respect of target structures that comprise target molecules bound by the target binding moieties of the proteins of the invention.

Preferably the cells killed by cytocidal activity of cells of the invention are cells associated with a disease. Suitably the cells associated with a disease may be cancer cells, or infected cells.

As set out in the Examples, the inventors have demonstrated that cells of the invention (comprising proteins of the invention) exhibit cytocidal activity that is directed to cells expressing target molecules bound by the target binding moieties of the proteins of the invention.

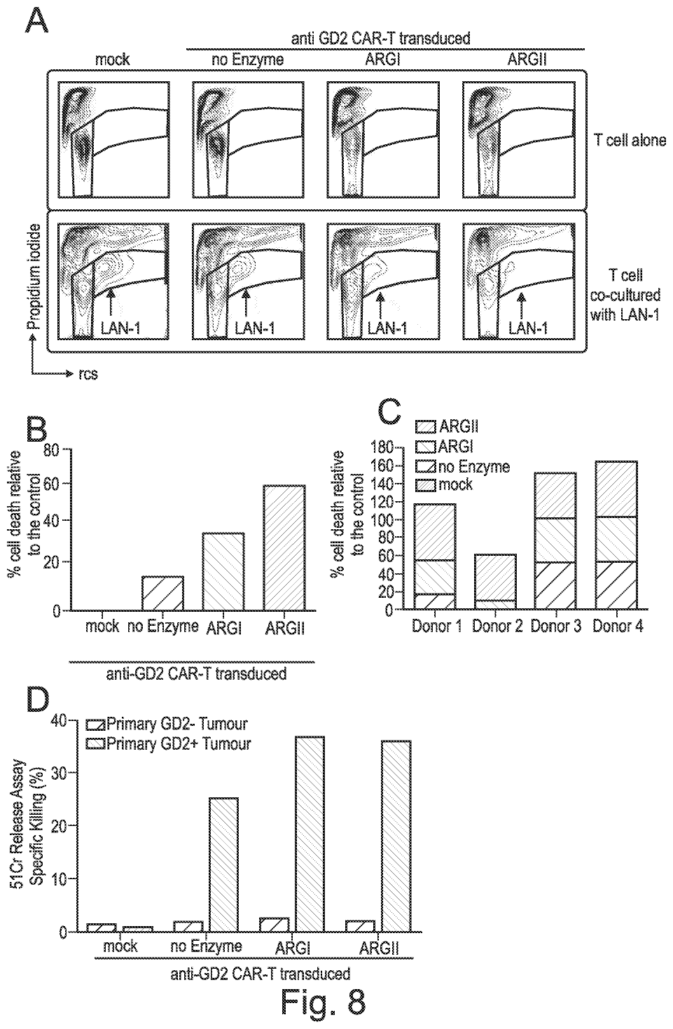

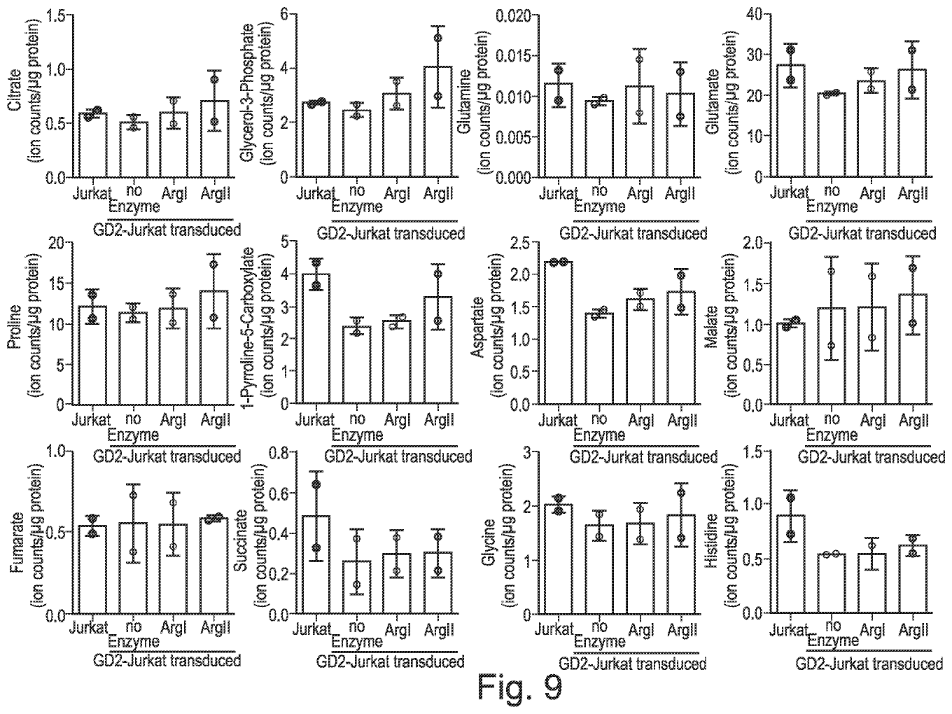

Cells of the invention expressing proteins of the invention comprising either an arginase type I or arginase type II domain have particularly improved cytocidal activity, as compared to control CAR-T cells. This improved cytocidal activity can be seen in the results set out in FIG. 8, which indicate that cells of the invention achieve level of target cell death that is around two to three times higher than that of control CARs without arginase enzyme domains. It will be appreciated that a two- or three-fold increase in cytocidal activity of this sort will be expected to confer marked benefits in terms of therapeutic effectiveness of the cells of the invention as compared to prior art CAR therapies.

Cells of the invention expressing proteins of the invention comprising either an arginase type I or arginase type II domain have particularly improved cytocidal activity, as compared to control CAR-T cells, in experimentally arginine-depleted conditions. This effect was most notable in cells of the invention comprising an arginase I domain, indicating a particular utility for these cells in application where local arginine levels are likely to be reduced, such as in the treatment of solid tumours.

Cells of the invention expressing proteins of the invention comprising an arginase type I or arginase type II domain also have improved cytocidal activity, as compared to control CAR-T cells, in conditions representative of the blood (“standard” or “arginine supplemented” conditions). This effect was particularly notable in respect of cells comprising an arginase II domain, indicating particular suitability of these cells in the treatment of blood-borne disease and cancers of the blood.

This increased cytocidal activity exhibited by the cells of the invention confers benefits not noted in respect of the cells of the prior art. In combination with the improved proliferation noted in respect of cells of the invention, it will be apparent to the skilled person that the proteins and cells disclosed herein have surprisingly advantageous therapeutic utility.

The skilled person will be aware of many suitable assays by which the cytocidal activity, whether cytotoxic activity or specific cell lysis, of a cell of the invention, or suitable comparator cell, may be assessed. Merely by way of example suitable assays are described in the Examples, where they are used in the characterisation of exemplary cells of the invention.

The skilled reader, on considering the information set out in the Examples, will recognise that the cells of the invention exhibit cytocidal activity that makes them well suited to therapeutic use in the prevention and/or treatment of disease such as cancer in the manner described in this specification.

Proliferation of Cells of the Invention

Activation of cells of the invention, via binding of the protein to the corresponding target molecule, induces cell proliferation, as demonstrated in the results set out in the Examples. This allows the production of increased numbers of cells able to exert a therapeutic activity. However, such cell proliferation is normally inhibited in the tumour microenvironment, and this has contributed to the failure of CAR T cell treatments disclosed in the prior art.

The cells of the invention exhibit proliferation capacity that is remarkably improved as compared to that observed in respect of CAR T cells of the prior art. This is particularly noted when proliferation of cells of the invention comprising arginase domains (whether arginase type I or arginase type II domains) is tested in conditions that replicate the arginine levels found in the blood (“standard” or “arginine supplemented” conditions). Since cell proliferation in the blood results in expansion of populations of cells of the invention that are then able to exert their therapeutic cytocidal activity either in the blood or within the tumour microenvironment, this is highly advantageous.

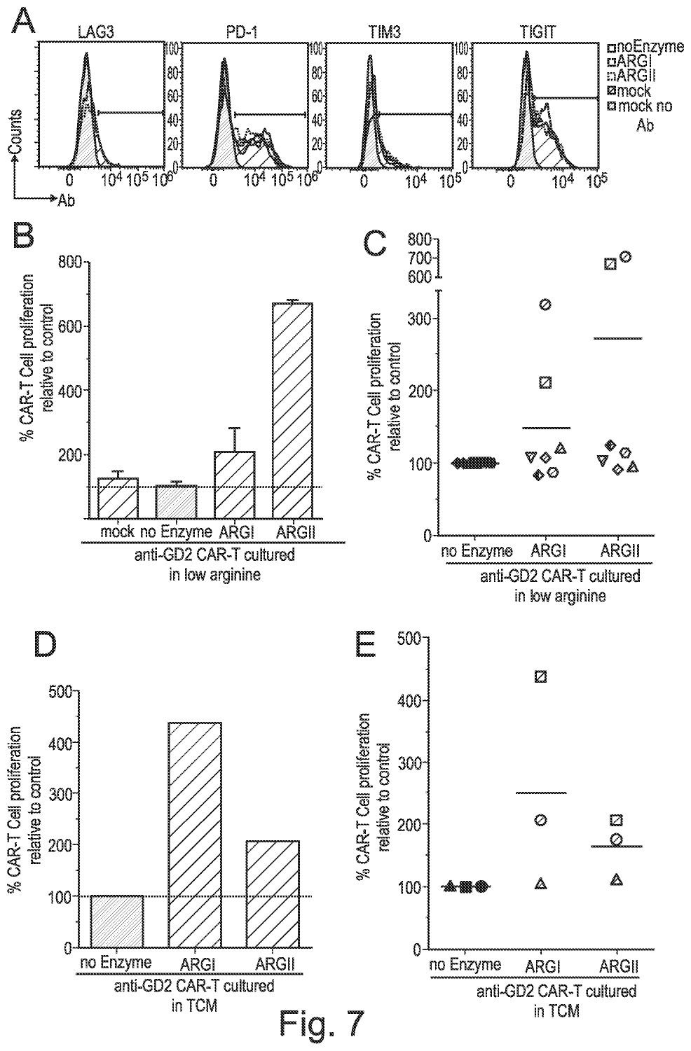

Furthermore, the results set out in FIG. 7 demonstrate that cells expressing the proteins of the invention exhibit markedly increased proliferation in low arginine culture conditions, or when cultured in tumour conditioned medium, as compared to cells expressing control CARs without arginase enzyme domains. These culture conditions (low arginine or tumour conditioned medium) provide well regarded experimental models of the suppressive tumour microenvironment, and so the results set out in the present specification clearly indicate improved activity of cells of the invention in this milieu. Cells expressing proteins of the invention comprising an arginase type I domain are particularly effective in terms of increased proliferation when cultured in tumour conditioned medium, while cells expressing proteins of the invention comprising an arginase type II domain demonstrate particularly increased proliferation under low arginine culture conditions.

Proliferation of cells, such as cells of the invention may be assessed experimentally in a number of ways. Merely by way of example, cell proliferation may be assessed in conditions that replicate the tumour microenvironment, or that replicate those found in the blood.

In suitable experimental conditions, such as those representative of the tumour microenvironment or the blood, the cells of the invention may exhibit a degree of proliferation that is at least 5% higher than that of suitable comparator cells, at least 10% higher than that of suitable comparator cells, at least 15% higher than that of suitable comparator cells, at least 20% higher than that of suitable comparator cells, at least 25% higher than that of suitable comparator cells, at least 30% higher than that of suitable comparator cells, at least 35% higher than that of suitable comparator cells, at least 40% higher than that of suitable comparator cells, at least 45% higher than that of suitable comparator cells, at least 50% higher than that of suitable comparator cells, at least 55% higher than that of suitable comparator cells, at least 60% higher than that of suitable comparator cells, at least 65% higher than that of suitable comparator cells, at least 70% higher than that of suitable comparator cells, at least 75% higher than that of suitable comparator cells, at least 80% higher than that of suitable comparator cells, at least 85% higher than that of suitable comparator cells, at least 90% higher than that of suitable comparator cells, or at least 95% higher than that of suitable comparator cells. Indeed, the cells of the invention may exhibit a degree of proliferation that is at least 100%, or more, higher than that of suitable comparator cells in the same experimental conditions. For example, and as demonstrated in the accompanying results, the cells of the invention may exhibit a degree of proliferation that is increased at least two-fold as compared to that of suitable comparator cells in the same experimental conditions. Indeed, the cells of the invention may exhibit a degree of proliferation that is increased at least three-fold, at least four-fold, at least five-fold, at least six-fold, at least seven-fold, at least eight-fold, at least nine-fold, or at least ten-fold as compared to that of suitable comparator cells in the same experimental conditions.

Alternatively, proliferation of cells may be assessed with reference to the number of cells present in a recipient after a set period of time from administration, as compared to the number of comparator cells present under the same conditions. The number of cells of the invention present in a recipient after a given time may be at least 5% higher than that of suitable comparator cells, at least 10% higher than that of suitable comparator cells, at least 15% higher than that of suitable comparator cells, at least 20% higher than that of suitable comparator cells, at least 25% higher than that of suitable comparator cells, at least 30% higher than that of suitable comparator cells, at least 35% higher than that of suitable comparator cells, at least 40% higher than that of suitable comparator cells, at least 45% higher than that of suitable comparator cells, at least 50% higher than that of suitable comparator cells, at least 55% higher than that of suitable comparator cells, at least 60% higher than that of suitable comparator cells, at least 65% higher than that of suitable comparator cells, at least 70% higher than that of suitable comparator cells, at least 75% higher than that of suitable comparator cells, at least 80% higher than that of suitable comparator cells, at least 85% higher than that of suitable comparator cells, at least 90% higher than that of suitable comparator cells, or at least 95% higher than that of suitable comparator cells if both cells of the invention and comparator cells are administered in approximately equal amounts.

Persistence of Cells of the Invention

The increased proliferation of cells of the invention, and hence greater number of cells available, will also be expected to improve persistence of the cells within a subject receiving treatment.

Medical Uses and Methods of Treatment

The proteins of the invention, particularly in the form of cells of the second aspect of the invention that comprise such proteins, are well suited to medical use, which is to say for use as medicaments in the prevention and/or treatment of diseases. Such medical uses and methods of treatment are the subject matter of the fifth and seventh aspects of the invention.

Prevention of a disease may be required when a subject has not yet developed a disease, but has been identified as being at risk of developing the disease in future. Suitably such identification may be based upon details such as the clinical history of the subject or their family, results of genetic testing of the subject of their family, or exposure risk to known disease causing agents. In the case of cancer, prevention may be desirable in the case of a subject exhibiting symptoms or features of pre-malignant disease.

Treatment of a disease may be required once a subject has been identified as already having developed a disease. The stage of development of the disease at the time of identification may be symptomatic or asymptomatic. Such identification may be based upon clinical assessment of the subject, symptoms presented by the subject, or analysis of samples provided by the subject (such biopsies, blood samples, or the like, allowing for the identification of the presence of malignancies, infectious agents, or other indicators of pathology).

The sixth aspect of the invention relates to a protein of the first aspect of the invention, a cell of the second aspect of the invention, a nucleic acid of the third aspect of the invention, or a pharmaceutical composition of the fourth aspect of the invention for use in the prevention and/or treatment of a disease. The prevention and/or treatment may be by targeted killing of blood borne cells. Suitably such blood borne cells may be blood borne cancer cells. Suitably such blood borne cells may be infected cells in the circulation. Further considerations regarding such medical uses in the prevention and/or treatment of cancer are set out elsewhere in the specification.

The seventh aspect of the invention relates to a method of preventing and/or treating a disease in a subject in need of such prevention and/or treatment, the method comprising providing the subject with a protein of the invention. The protein of the invention is provided in a therapeutically effective amount. Such a therapeutically effective amount may be achieved by a single incidence of providing a protein of the invention, or cumulatively through multiple incidences of providing proteins of the invention.

The protein of the invention may suitably be provided to the subject directly or indirectly. By direct provision is meant the administration of the protein, particularly in the form of a cell expressing the protein, to the subject. By indirect provision is meant inducing the subject to express a protein of the invention. It will be appreciated that a protein of the invention may be provided indirectly to a subject via administration of a nucleic acid of the third aspect of the invention, which encodes a protein according to the first aspect of the invention.

As with the medical uses of the sixth aspect of the invention, the methods of the seventh aspect of the invention may be used in preventing and/or treating a disease by targeted killing of blood borne cells. The blood borne cells may be blood borne cancer cells or infected cells in the circulation, as before.

While the cells of the invention exhibit desirable biological activities under conditions that replicate the immunosuppressive tumour microenvironment, and so are particularly suitable for the prevention and/or treatment of cancer (as discussed further below), their improved cytocidal activity is also demonstrated in conditions in which arginine is not depleted. Accordingly, it will be appreciated that cells comprising proteins of the invention may be used in the prevention or treatment of a wide range of diseases, including not just cancers, but also autoimmune diseases and diseases caused by infections, such as viral infections. Suitably such diseases may be prevented and/or treated by medical uses of methods of treatment utilising the proteins, cells, nucleic acids, or pharmaceutical compositions of the invention.

As set out elsewhere in the specification, cells of the invention may be for use in autologous treatment, or for use in heterologous treatment.

Prevention and/or Treatment of Cancer

In particular, the proteins, cells, nucleic acids, or pharmaceutical compositions of the invention may be of use in the prevention and/or treatment of cancer. It is in these applications that the cells of the invention's increased cytocidal and proliferative activity (as compared to control CAR-T cells) under arginine-depleted conditions representative of the tumour microenvironment are particularly advantageous.

Suitable examples of cancers that may be prevented and/or treated by medical uses or methods of treatment utilising the proteins, cells, nucleic acids, or pharmaceutical compositions of the invention include those associated with cancer cell expression of one or more markers selected from the group consisting of: GD2; CD33; Mesothelin; EGFRvIII; VEGFR2; FAP; EpCam; GPC3; CD133; IL13Ra; EphA2; Muc1; BCMA; CD70; CD123; ROR1; PSMA; CD5; GAP; CEA; PSCA; Her2; and CD19. Such cancers may be treated by use of fusion target-binding proteins of the invention incorporating corresponding target binding moieties.

Merely by way of example, suitable cancers that may be prevented and/or treated by medical uses or methods of treatment of the invention may be selected from the group consisting of: neuroblastoma; mesothelioma; ovarian cancer; breast cancer; colon cancer; medulloblastoma; pancreatic cancer; prostate cancer; testicular cancer; acute myeloid leukaemia; glioblastoma; osteosarcoma; and melanoma.

Pharmaceutical Compositions and Formulations

The present invention also provides compositions including proteins, cells, or nucleic acids of the invention. In particular, the invention provides pharmaceutical compositions and formulations, such as unit dose form compositions including proteins, cells, or nucleic acids of the invention for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some embodiments, the choice of carrier is determined in part by the particular protein, cell; or nucleic add of the invention, and/or by the method of administration. Accordingly, there are a variety of suitable formulations. For example, the pharmaceutical composition can contain preservatives. Suitable preservatives may include, for example, methylparaben, propylparaben, sodium benzoate; and benzalkonium chloride. In some aspects, a mixture of two or more preservatives is used. The preservative or mixtures thereof are typically present in an amount of about 0.0001 to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatine, or immunoglobulins; hydrophilic polymers such as polyvinylpyrrolidone; amino acids such as glycine, glutamine, asparagine, histidine, arginine, or lysine; monosaccharides, disaccharides; and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents are included in some embodiments of the compositions of the invention. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001 to about 4% by weight of the total composition. Methods for preparing administrable pharmaceutical compositions are known. Exemplary methods are described in more detail in, for example, Remington: The Science and Practice of Pharmacy; Lippincott Williams & Wilkins; 21st ed. (May 1, 2005).

The formulations can include aqueous solutions. The formulation or composition may also contain more than one active ingredient useful for the particular indication, disease, or condition being treated with the proteins s, cells, or nucleic acids of the invention, preferably those with activities complementary to the proteins s; cells; or nucleic acids of the invention, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g.; asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the CARs, cells, or nucleic acids of the invention in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the proteins, cells, or nucleic acids of the invention, by multiple bolus administrations of the proteins, cells, or nucleic acids, or by continuous infusion administration of the proteins, cells, or nucleic acids.

The compositions may be administered using standard administration techniques, formulations, and/or devices. Administration of the cells can be autologous or heterologous. For example, immunoresponsive cells or progenitors can be obtained from one subject, and administered to the same subject (autologous) or a different, compatible subject (heterologous). Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the proteins, cells, or nucleic acids of the invention in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavouring agents, and/or colours, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminium monostearate and gelatine.

The formulations to be used for in vivo administration are generally sterile. Sterility may be readily accomplished, e.g., by filtration through sterile filtration membranes.

Doses and Dosage Regimens

Size or Amount of Doses

The proteins, cells, or nucleic acids of the invention may be provided in a first dose, and optionally in subsequent doses. In some embodiments, the first or subsequent dose contains a number of proteins, cells, or nucleic acids of the invention in the range from about 10⁵ to about 10⁶ of such cells per kilogram body weight of the subject, and/or a number of such cells that is no more than about 10⁵ or about 10⁶ such cells per kilogram body weight of the subject. For example, in some embodiments, the first or subsequent dose includes less than or no more than at or about 1×10⁵, at or about 2×10⁵, at or about 5×10⁵, or at or about 1×10⁶ of such cells per kilogram body weight of the subject. In some embodiments, the first dose includes at or about 1×10⁵, at or about 2×10⁵, at or about 5×10⁵, or at or about 1×10⁶ of such cells per kilogram body weight of the subject, or a value within the range between any two of the foregoing values.

In some embodiments, for example, where the subject is a human, the first or subsequent dose includes fewer than about 1×10 total proteins, cells, or nucleic acids of the invention e.g., in the range of about 1×10⁶ to 1×10⁶ such cells, such as 2×10⁶, 5×10⁶, 1×10⁷, 5×10⁷, or 1×10⁸ or total such cells, or the range between any two of the foregoing values.

In some embodiments, the first or subsequent dose contains fewer than about 1×10⁵ total proteins, cells, or nucleic acids of the invention per m² of the subject, e.g., in the range of about 1×10⁶ to 1×10 8 such cells per m 2 of the subject, such as 2×106, 5×106, 1×107, 5×107, or 1×108 such cells per m of the subject, or the range between any two of the foregoing values.

In certain embodiments, the number of proteins, cells, or nucleic acids of the invention in the first or subsequent dose is greater than about 1×10⁶ such proteins, cells, or nucleic acids of the invention per kilogram body weight of the subject, e.g., 2×10⁶, 3×10⁶, 5×10⁶, 1×10⁷, 5×10⁷, 1×10⁸, 1×10⁹, or 1×10¹⁰ such cells per kilogram of body weight and/or, 1×108, or 1×109, 1×1010 such cells per m 2 of the subject or total, or the range between any two of the foregoing values.

In some embodiments, the number of proteins, cells, or nucleic acids of the invention administered in the subsequent dose is the same as or similar to the number of proteins, cells, or nucleic acids of the invention administered in the first dose in any of the embodiments herein, such as less than or no more than at or about 1×10⁵, at or about 2×10⁵, at or about 5×10⁵, or at or about 1×10⁶ of such cells per kilogram body weight of the subject. In some embodiments, the subsequent dose(s) contains at or about 1×10⁵, at or about 2×10⁵, at or about 5×10⁵, or at or about 1×10⁶ of such cells per kilogram body weight of the subject, or a value within the range between any two of the foregoing values. In some embodiments, such values refer to numbers of proteins, cells, or nucleic acids of the invention. In some aspects, the subsequent dose is larger than the first dose. For example, in some embodiments, the subsequent dose contains more than about 1×10⁶ proteins, cells, or nucleic acids of the invention per kilogram body weight of the subject, such as about 3×10⁶, 5×10⁶, 1×107, 1×108, or 1×109 such cells per kilogram body weight of the subject. In some embodiments, the amount or size of the subsequent dose is sufficient to reduce disease burden or an indicator thereof, and/or one or more symptoms of the disease or condition. In some embodiments, the second (or other subsequent) dose is of a size effective to improve survival of the subject, for example, to induce survival, relapse-free survival, or event-free survival of the subject for at least 6 months, or at least 1, 2, 3, 4, or 5 years. In some embodiments, the number of proteins, cells, or nucleic acids of the invention administered and/or number of such cells administered per body weight of the subject in the subsequent dose is at least 2-fold, 5-fold, 10-fold, 50-fold, or 100-fold or more greater than the number administered in the first dose. In some embodiments, disease burden, tumour size, tumour volume, tumour mass, and/or tumour load or bulk is reduced following the subsequent dose by at least at or about 50, 60, 70, 80, 90% or more compared to that immediately prior to the administration of the first dose or of the second (or other subsequent) dose.

In other embodiments, the number of proteins, cells, or nucleic acids of the invention administered in the subsequent dose is lower than the number of proteins, cells, or nucleic acids of the invention administered in the first dose.

In some embodiments, multiple subsequent doses are administered following the first dose, such that an additional dose or doses are administered following administration of the second (or other subsequent) dose. In some aspects, the number of cells administered to the subject in the additional subsequent dose or doses (i.e., the third, fourth, fifth, and so forth) is the same as or similar to the first dose, the second dose, and/or other subsequent dose. In some embodiments, the additional dose or doses are larger than prior doses.

In some aspects, the size of the first and/or subsequent dose is determined based on one or more criteria such as response of the subject to prior treatment, e.g. chemotherapy, disease burden in the subject, such as tumour load, bulk, size, or degree, extent, or type of metastasis, stage, and/or likelihood or incidence of the subject developing toxic outcomes, e.g., CRS, macrophage activation syndrome, tumour lysis syndrome, neurotoxicity, and/or a host immune response against the cells and/or recombinant receptors being administered.

In some aspects, the size of the first and/or subsequent dose is determined by the burden of the disease or condition in the subject. For example, in some aspects, the number of proteins, cells, or nucleic acids of the invention administered in the first dose is determined based on the tumour burden that is present in the subject immediately prior to administration of the first dose. In some embodiments, the size of the first and/or subsequent dose is inversely correlated with disease burden. In some aspects, as in the context of a large disease burden, the subject is administered a low number of proteins, cells, or nucleic acids of the invention, for example less than about 1×10⁶ proteins, cells, or nucleic acids of the invention per kilogram of body weight of the subject. In other embodiments, as in the context of a lower disease burden, the subject is administered a larger number of proteins, cells, or nucleic acids of the invention, such as more than about 1×10⁶ proteins, cells, or nucleic acids of the invention per kilogram body weight of the subject.

In some aspects, the number of proteins, cells, or nucleic acids of the invention administered in the subsequent dose is determined based on the tumour burden that is present in the subject following administration of the first dose. In some embodiments, e.g. where the first dose has decreased disease burden or has done so below a particular threshold amount or level, e.g., one above which there is an increased risk of toxic outcome, the subsequent dose is large, e.g. more than 1×10⁶ proteins s, cells, or nucleic acids of the invention per kilogram body weight, and/or is larger than the first dose. In other aspects, the number of proteins, cells, or nucleic acids of the invention administered in the subsequent dose is low, e.g. less than about 1×10⁶, e.g. the same as or lower than the first dose, where the first dose has reduced tumour burden to a small extent or where the first dose has not led to a detectable reduction in tumour burden.

In some embodiments, the number of proteins, cells, or nucleic acids of the invention administered in the first dose is lower than the number of proteins, cells, or nucleic acids of the invention administered in other methods, such as those in which a large single dose of cells is administered, such as to administer the proteins, cells, or nucleic acids of the invention in before an immune response develops. Thus, in some embodiments, the methods reduce toxicity or toxic outcomes as compared to other methods that involve administration of a larger dose.

In some embodiments, the first dose includes the proteins, cells, or nucleic acids of the invention in an amount that does not cause or reduces the likelihood of toxicity or toxic outcomes, such as cytokine release syndrome (CRS), severe CRS (sCRS), macrophage activation syndrome, tumour lysis syndrome, fever of at least at or about 38 degrees Celsius for three or more days and a plasma level of CRP of at least at or about 20 mg/dL, and/or neurotoxicity. In some aspects, the number of cells administered in the first dose is determined based on the likelihood that the subject will exhibit toxicity or toxic outcomes, such as CRS, sCRS, and/or CRS-related outcomes following administration of the cells. For example, in some embodiments, the likelihood for the development of toxic outcomes in a subject is predicted based on tumour burden. In some embodiments, the methods include detecting or assessing the toxic outcome and/or disease burden prior to the administration of the dose.

In some embodiments, the second (or other subsequent) dose is administered at a time point at which a clinical risk for developing cytokine-release syndrome (CRS), macrophage activation syndrome, or tumour lysis syndrome, or neurotoxicity is not present or has passed or has subsided following the first administration, such as after a critical window after which such events generally have subsided and/or are less likely to occur, e.g., in 60, 70, 80, 90, or 95% of subjects with a particular disease or condition.

Timing of Doses

In some aspects, the timing of the second or subsequent dose is measured from the initiation of the first dose to the initiation of the subsequent dose. In other embodiments, the timing of the subsequent dose is measured from the completion of the first dose, or from the median day of administration of the first dose, e.g. in the context of split dosing, described herein, where a dose is administered over more than one day, e.g. over 2 days or over 3 days.

In some embodiments, whether a subsequent dose of proteins, cells, or nucleic acids of the invention distinct from that of the first dose is administered, is determined based on the presence or degree of an immune response or detectable immune response in the subject to the proteins, cells, or nucleic acids of the invention of the first dose. In some aspects, a subsequent dose containing cells expressing a different receptor than the cells of the first dose will be administered to a subject with a detectable host adaptive immune response, or an immune response that has become established or reached a certain level, stage, or degree.

In some embodiments, the second (or other subsequent) dose is administered at a point in time at which a second administration of proteins, cells, or nucleic acids of the invention is likely to be or is predicted to be eliminated by the host immune system. The likeliness of developing an immune response may be determined by measuring receptor-specific immune responses in the subject following administration of the first dose, as described herein.

For example, in some embodiments, subjects may be tested following the first (or other prior) dose and prior to the second (or other subsequent) dose to determine whether an immune response is detectable in the subject after the first dose. In some such embodiments, the detection of an immune response to the first dose may trigger the need to administer the second dose.

In some aspects, samples from the subjects may be tested to determine if there is a decline in or lower than desired exposure, for example, less than a certain number or concentration of cells, as described herein, in the subject after the first or prior dose. In some such aspects, the detection of a decline in the exposure of the subject to the cells may trigger the need to administer the second dose.

In some embodiments, the subsequent dose is administered at a point in time at which the disease or condition in the subject has not relapsed following the reduction in disease burden in response to the first or prior dose. In some embodiments, the disease burden reduction is indicated by a reduction in one or more factors, such as load or number of disease cells in the subject or fluid or organ or tissue thereof, the mass or volume of a tumour, or the degree or extent of metastases. Such a factor is deemed to have relapsed if after reduction in the factor in response to an initial treatment or administration, the factor subsequently increases.

In some embodiments, the second dose is administered at a point in time at which the disease has relapsed. In some embodiments, the relapse is in one or one or more factors, or in the disease burden generally. In some aspects, the subsequent dose is administered at a point in time at which the subject, disease burden, or factor thereof has relapsed as compared to the lowest point measured or reached following the first or prior administration, but still is lower compared to the time immediately prior to the first dose. In some embodiments, the subject is administered the subsequent dose at a point in time at which disease burden or factor indicative thereof has not changed, e.g. at a time when an increase in disease burden has been prevented.

In some embodiments, the subsequent dose is administered at a time when a host adaptive immune response is detected, has become established, or has reached a certain level, degree, or stage. In some aspects, the subsequent dose is administered following the development of a memory immune response in the subject.

In some aspects, the time between the administration of the first dose and the administration of the subsequent dose is about 28 to about 35 days, about 29 to about 35 days, or more than about 35 days. In some embodiments, the administration of the second dose is at a time point more than about 28 days after the administration of the first dose. In some aspects, the time between the first and subsequent dose is about 28 days.

In some embodiments, an additional dose or doses, e.g. subsequent doses, are administered following administration of the second dose. In some aspects, the additional dose or doses are administered at least about 28 days following administration of a prior dose. In some embodiments, no dose is administered less than about 28 days following the prior dose.

In some embodiments, e.g. where one or more consecutive doses are administered to the subject, the consecutive doses may be separated by about 7, about 14, about 15, about 21, about 27, or about 28 days. In some aspects, the consecutive dose is administered 21 days following a prior dose. In some embodiments, the consecutive dose is administered between 14 and 28 days following administration of a prior dose,

In any of the embodiments, the methods in some cases include the administration of the first or prior dose and the subsequent dose(s), and in other cases include the administration of the subsequent dose(s) to a subject who has previously received the first or prior dose but do not include the administration of the first or prior dose itself. Thus, the methods in some cases involve the administration of consolidating treatment, such as by administering a consolidating subsequent dose to a subject that has previously received a dose of proteins, cells, or nucleic acids of the invention.

In some embodiments, disease burden, tumour size, tumour volume, tumour mass, and/or tumour load or bulk is reduced following the subsequent dose by at least at or about 50, 60, 70, 80, 90% or more compared to that immediately prior to the administration of the first or prior dose or of the second or subsequent dose.

Manufacture of Cells Comprising Proteins of the Invention

The skilled person will be aware of suitable methods by which nucleic acids, such as the nucleic acids of the invention, may be used in the manufacture of transduced cells expressing proteins. Such methods may be used in the manufacture of cells of the invention, which express proteins of the invention.

In the fifth aspect of the invention a cell of the second aspect of the invention is manufactured by a method comprising providing a cell with a nucleic acid molecule of the third aspect of the invention. This is performed under conditions such that the nucleic acid molecule is expressed by the cell to produce a fusion target-binding protein in accordance with the first aspect of the invention.

Suitably the cell may be a leukocyte, such as a T cell. Methods that are conventional in the manufacture of CAR-T cells may be utilised in the methods of the sixth aspect of the invention. Suitable methods may involve some or all of the following steps: T cell selection; T cell activation; provision of the nucleic acid to activated T cells; expansion of T cell numbers; and formulation into a pharmaceutical composition in accordance with the fourth aspect of the invention. Such a composition may then be preserved, for example by cryopreservation, until provided to a subject requiring treatment.

Merely by way of example, suitable protocols that may be used in the manufacture of cells of the invention are described further in the Examples below.

Other examples of methods for the manufacture of cells expressing proteins of the invention will be apparent to those skilled in the art. Without limitation, these include methods by which nucleic acids of the invention are introduced to cells by means such as viruses or nanoparticles.

### EXAMPLES 1

The proteins of the invention were investigated with reference to exemplary CARs, as discussed further below.

1.1 Optimisation of CAR-Containing Viral Titres.

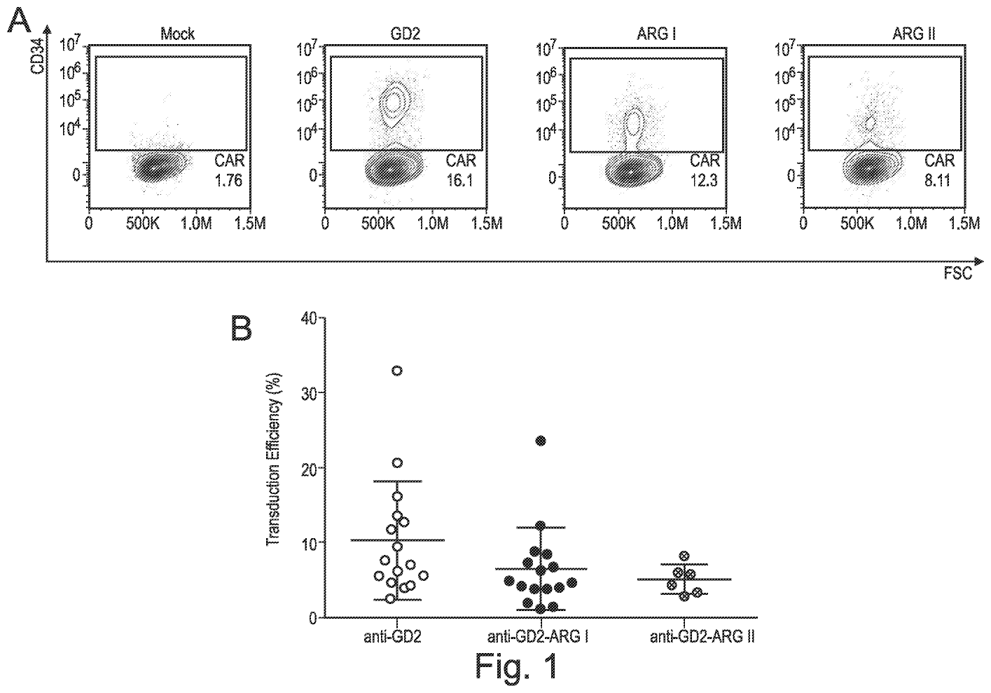

Transduction efficiency of nucleic acid sequences encoding proteins of the invention comprising either arginase type I or arginase type II domains was assessed by flow cytometry detection of tCD34. No significant difference in transduction efficiency of PBMCs (either human T cells, or cells of the Jurkat cell line) was seen, as shown in FIG. 1. In this Figure, panel A illustrates representative flow cytometry staining, while panel B sets out a summary of transduction efficiency across multiple T cell donors.

1.2 Proteins of the Invention Comprising Arginase Type I and Arginase Type II Domains Retain Arginase Activity in Transduced Human T Cells and Jurkat Cells.

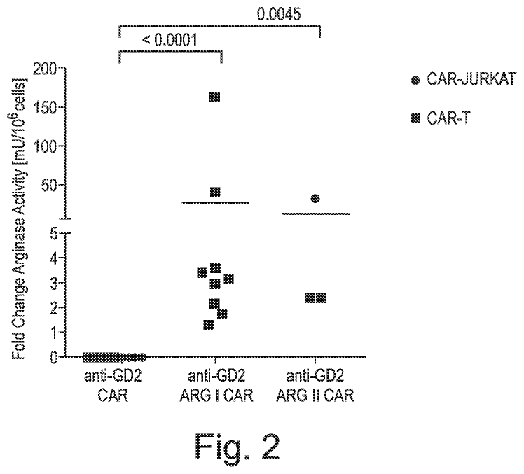

Results set out in FIG. 2 illustrate the ability of the arginase domains present in fusion target-binding proteins of the invention expressed by transduced cells to perform their arginase function. The catabolism of arginine into ornithine and urea, by arginase type I or arginase type II domains, was assessed and compared to control constructs, without enzyme domains (GD2 only). It can be seen that proteins of the invention comprising both arginase type I and arginase type II domains provided significant increases in cellular arginase activity as compared to the controls.

1.3 Proteins of the Invention Significantly Enhance Cytocidal Activity of Transfected Cells In Vitro

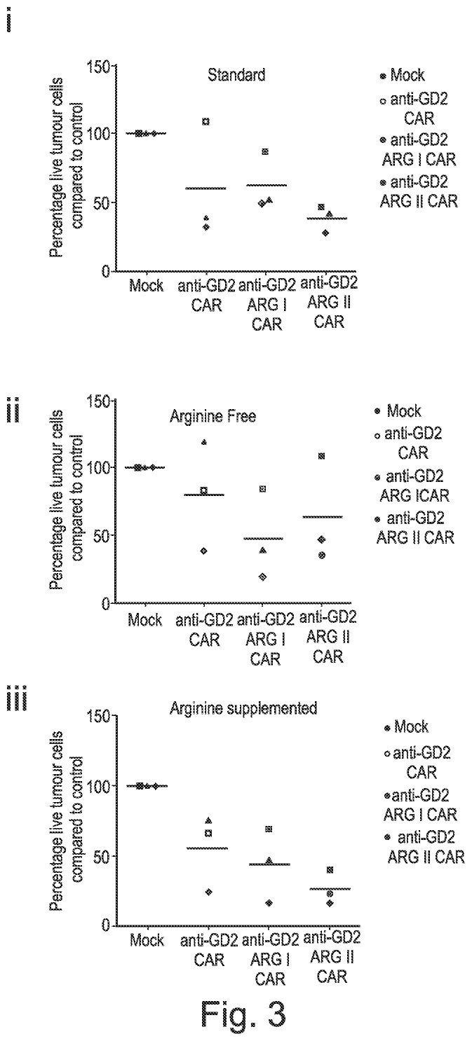

FIG. 3 illustrates specific cell lysis by T cells of the invention expressing fusion target-binding proteins comprising an arginase type I domain (GD2-ARG1), or an arginase type II domain (GD2-ARG2) on neuroblastoma cell line assessed against the control (GD2 only) under conditions of i) standard culture conditions, ii) arginine-free conditions, iii) supplemented arginine conditions.

It can be seen that the cells of the invention comprising arginase type I domains exhibited increased cytocidal activity (as compared to controls) in arginine free and arginine supplemented conditions, and exhibited comparable cytocidal activity under standard culture conditions. These cells had the highest cytocidal activity among those tested under arginine free conditions representative of the tumour microenvironment.

Cells of the invention comprising arginase type II domains exhibited increased cytocidal activity (as compared to controls) in all conditions tested. Among the cells investigated, these cells of the invention had the highest cytocidal activity under standard and arginine supplemented conditions representative of the conditions found in the blood.

1.4 Proliferation of Cells of the Invention Under Various Arginine Culture Conditions

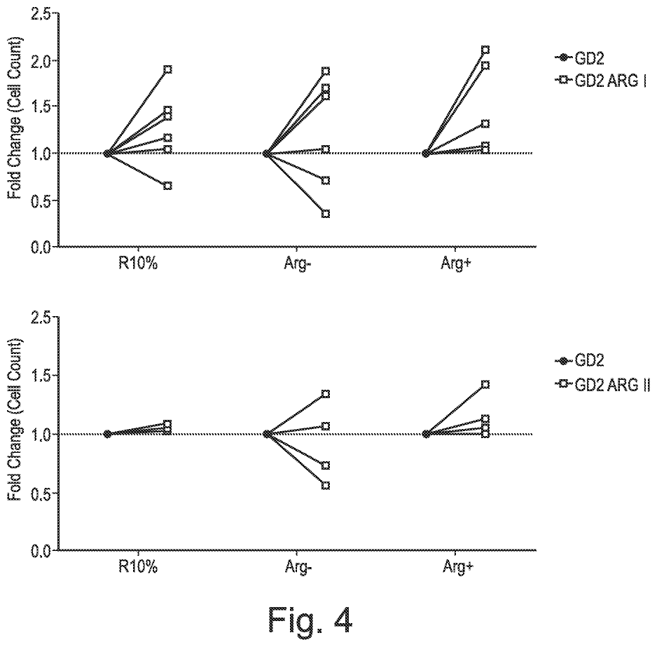

FIG. 4 shows T cells of the invention comprising fusion target-binding proteins comprising arginase type I or arginase type II domains showed enhanced proliferation compared to the control (GD2 only) in standard (R10%), arginine free (ARG−) and arginine supplemented (ARG+) conditions. The increase in proliferation (for both cells comprising arginase type I domains and cells comprising arginase type II domains) was most notable in arginase supplemented conditions, indicating that the cells of the invention will be able to proliferate in the blood, and then provide a reservoir of therapeutically effective cells (which may exert their cytocidal activity against targets present in the blood, or in other tissues of the body).

### EXAMPLES 2

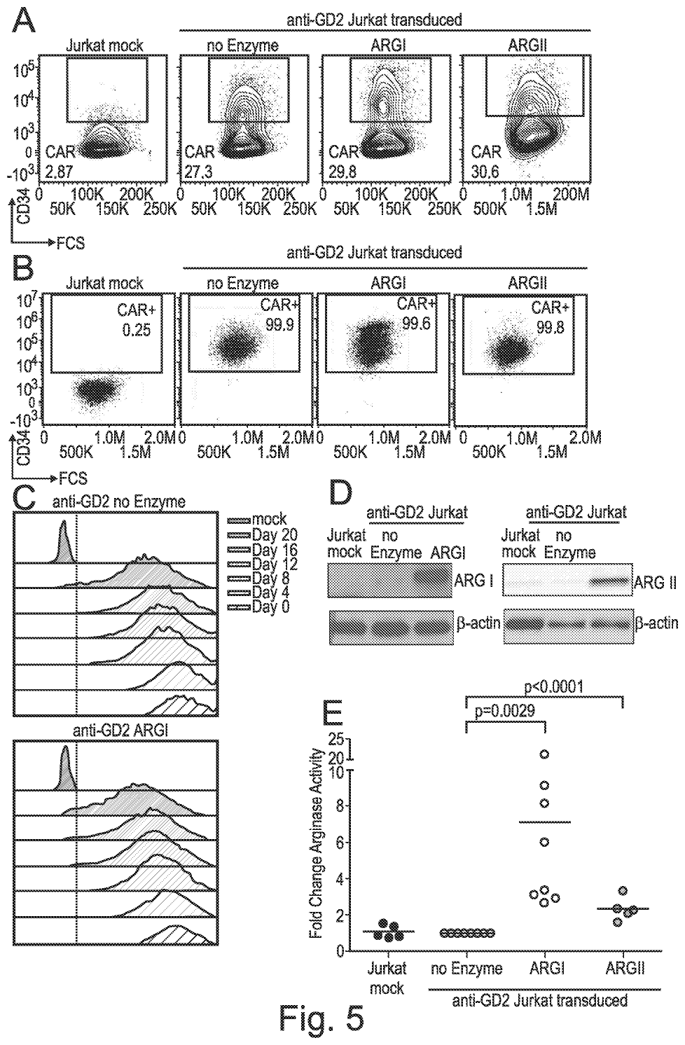

Further studies were undertaken to confirm and expand upon the results reported in the “Examples 1” section above. The results of these are set out in FIGS. 5 to 10.

The results set out in FIG. 5 confirm the finding that arginase I and II enzymes present in proteins of the invention demonstrate activity in transduced Jurkat T cells.

Panel A show the results of transduction of proteins of the invention comprising anti-GD2 CARs with either no enzyme domain, an arginase type I domain (ARG1) or an arginase type II domain (ARG2) into Jurkat T cell lines and sorted to a high degree of purity (Panel B) as assessed by measuring expression of tCD34 using flow cytometry. Panel C indicates the proteins of the invention are stable in cells post transduction over time. The results presented demonstrate that both control CARs (anti-GD2 only—no enzyme domain) and proteins of the invention comprising an anti-GD2 target binding moiety and an arginase type I domain (“anti-GD2 ARG1” could be detected within transduced cells over 20 days of culture in vitro. Panel D shows the expression of the arginase type I domain or arginase type II domains in proteins of the invention expressed in transduced cells. Panel E illustrates the ability of the arginase domains of proteins of the invention to perform their function (that is to say, to catabolise arginine) in transduced cells. The catabolism of arginine into ornithine and urea, by arginase type I or arginase type II domains of proteins of the invention, was assessed and compared to control constructs—Jurkat mock alone or anti-GD2 CARs without enzyme domains (“no enzyme”). It can be seen that arginase activity in cells transduced to express proteins of the invention (“ARG1” or “ARG2”) was significantly increased as compared to that demonstrated by controls (“Jurkat mock”—receiving no CAR, or “no Enzyme” transduced to express anti-GD2 CARs without arginase domains).

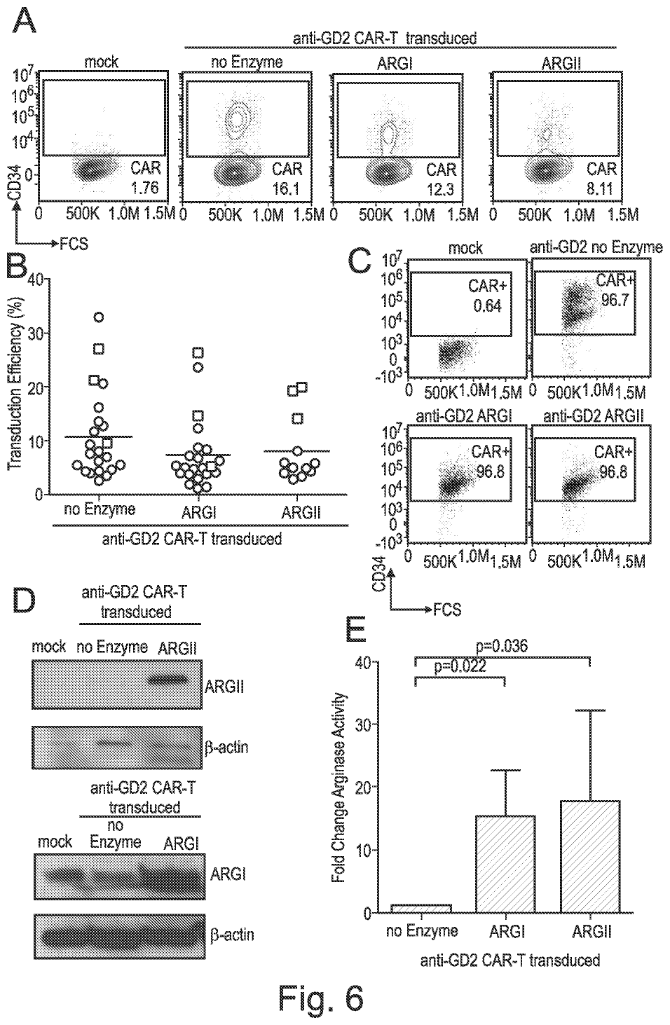

The results set out in FIG. 6 show that arginase type I and II domains in proteins of the invention demonstrate activity in human T cells.

The results shown in FIG. 6 confirm and expand upon those generated in the studies undertaken as part of Examples 1. Panel A show proteins of the invention can be transduced into human T cells from donors (panel shows representative flow cytometry staining, and reproduces the information set out in FIG. 1A). Panel B is a summary of transduction efficiency across multiple T cell donors and illustrates the same trends shown in FIG. 1B. Panel C illustrates that transduced human T cells can be sorted to a high degree of purity Panel D shows the expression of arginase type I or arginase type II domains in proteins of the invention expressed by the transduced cells. Panel E illustrates the ability of the arginase domains of proteins of the invention to functionally catabolise arginine in the transduced cells. The catabolism of arginine into ornithine and urea, by anti-GD2 target-binding proteins of the invention comprising either arginase type I or arginase type II domains, was assessed and compared to control constructs without enzyme domains (“no Enzyme”—anti-GD2 CARs that do not include arginase domains).

The results set out in FIG. 7 show that arginase I and II enzyme domains in proteins of the invention enhance T cell proliferation in low arginine and tumour conditions, and do not adversely affect T cell exhaustion.

FIG. 7 sets out the results of a study confirming, and expanding upon, the results shown in FIG. 4. Panel A of FIG. 7 shows that transduction of protein of the invention comprising either arginase type I or type II domains into T cells does not increase the expression of exhaustion markers LAG3, PD-1, TIM3, or TIGIT, as assessed by flow cytometry compared to controls. T cells expressing proteins of the invention comprising either arginase type I or type II domains (“ARG-I” or “ARG-II”) showed enhanced proliferation compared to the controls (mock or GD2 no enzyme only) in culture conditions with low arginine (Panels B and C) or when grown in tumour conditioned media (“TCM”—Panels D and E). These results reinforce the finding that cells expressing proteins of the invention are well suited for use as therapeutic agents. The lack of elevated exhaustion marker expression shown in FIG. 7A indicates that these cells will be capable of generating a vigorous and sustained response to cells expressing their target protein. The ability of the cells to proliferate in condition of reduced arginine or in tumour conditioned media indicate that they are well suited to overcome condition associated with the immunosuppressive tumour microenvironment, which otherwise contribute to poor clinical outcomes associated with the use of prior art CAR T cells in the treatment of solid tumours.

The results set out in FIG. 8 show that cells expressing proteins of the invention comprising arginase I and II enzyme domains have enhanced antigen-specific cytotoxicity against target tumour cells

Panels A and B of FIG. 8 illustrate specific cell lysis of T cells expressing exemplary fusion target-binding proteins of the invention comprising either arginase type I domains (“GD2-ARGI”), or arginase type II domains (“GD2-ARGII”). Cell lysis was demonstrated on neuroblastoma cell line LAN-1 and was assessed against the controls (mock or GD2 only) as assessed by flow cytometry. As can be seen both proteins of the invention comprising an arginase type I domain and proteins of the invention comprising an arginase type II domain demonstrated markedly increased killing of neuroblastoma target cells as compared to mock transduced or “no Enzyme” (anti-GD2 CAR without arginase domains) controls. Panel C illustrates the enhanced cytotoxicity of cells expressing proteins of the invention is demonstrated in T cells derived from four different donors. Panel D confirms transduced T cells retain their antigen-specific killing (illustrated by their level of killing against a GD2+ tumour and a GD2− tumour) and have greater cytocidal activity than T cells expressing anti-GD2 CARs without arginase domains (“no Enzyme” controls).

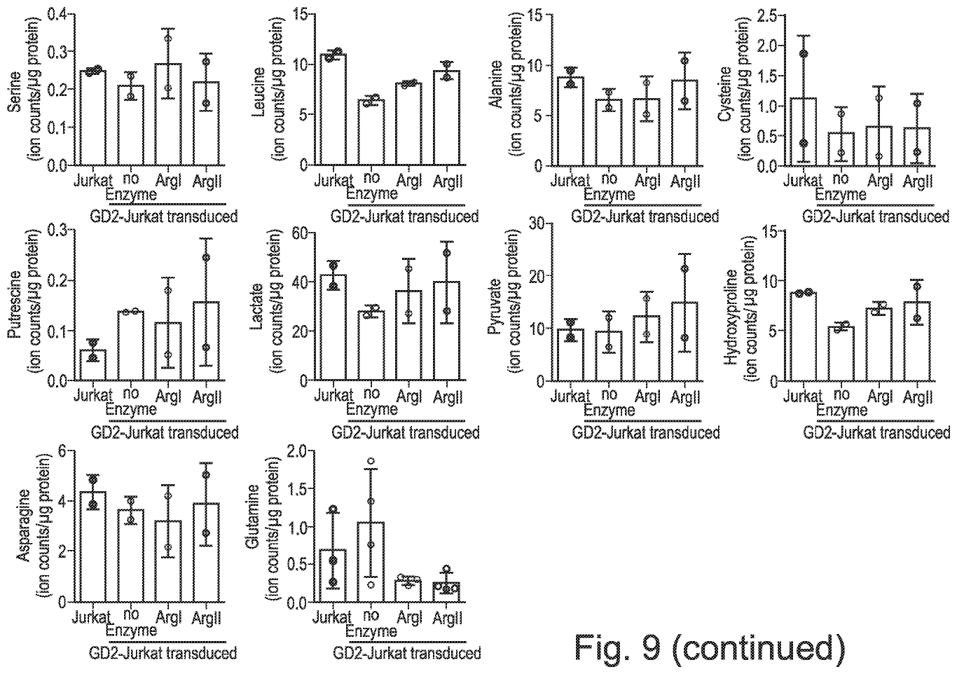

The results set out in FIG. 9 show that the presence of arginase enzyme domains in proteins of the invention leads to altered intracellular metabolic profiles

Jurkat T cells transduced to express proteins of the invention comprising either arginase type 1 or arginase type H domains were subjected to metabolic tracing. CAR-Jurkat were cultured for 48 h in phenol free SILAC RPMI 1640 Rex media supplemented with dialysed FBS (10%), 10 mM Glucose, 2 mM L-Glutamine, 200 uM L-Lysine, 100 uM L-Citrulline, 250 um L-Arginine (or U-¹³C L-Arginine) and 250 uM L-Ornithine (or U-¹³C L-Ornithine). GC-MS analysis was performed, and intracellular metabolites were normalised against sample protein content. Changes in intracellular metabolites are identified between cells transduced to express proteins of the invention with arginase enzyme domains and control cells.

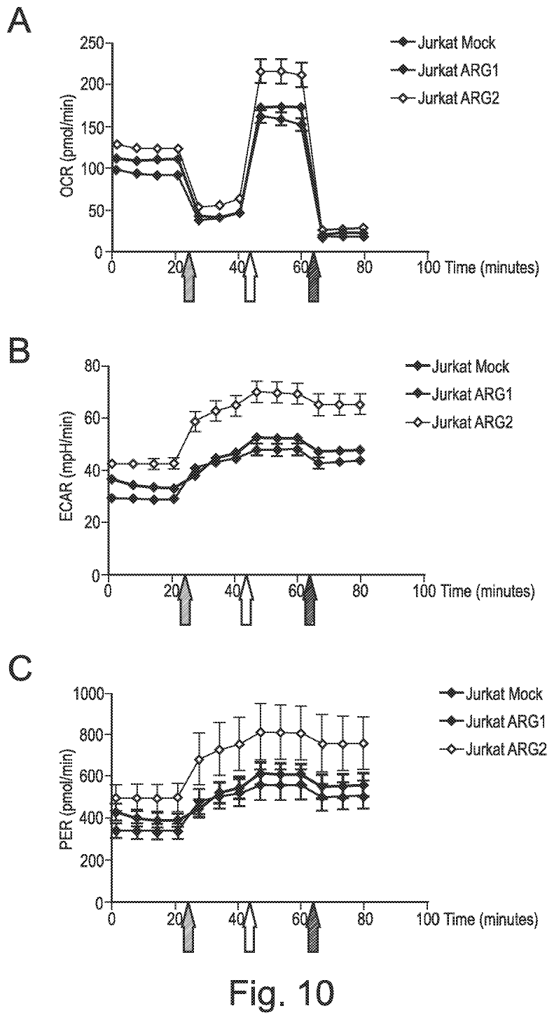

The results set out in FIG. 10 show that presence of arginase enzyme domains in proteins of the invention can lead to altered mitochondrial respiration

Jurkat T cells transduced to express proteins of the invention comprising arginase type I and arginase type II domains were subjected to a Seahorse XF Mito Stress Test. Oligomycin (2 uM), BAM15 (3 uM) and Rotenone & Antirnycin A (both 2 uM) were injected at 24 min, 44 min and 64 min respectively, to challenge the different steps of the electron transport chain. Oxygen consumption rate (OCR, Panel A), extracellular acidification rate (ECAR, Panel B) and proton efflux rate (PER, Panel C) were measured over the course of the assay.

Without wishing to be bound by any hypothesis, the inventors believe that the changes in intracellular metabolism and mitochondrial respiration demonstrated in FIGS. 9 and 10 contribute to the modified biological activities (including increased proliferation and cytolytic activity) that confer therapeutic advantages on cells expressing proteins of the invention.

Protocols for the Production of Cells of the Invention

Cells of the invention have been successfully produced by retroviral and by lentiviral transduction approaches. Details of an exemplary protocol for the retroviral production of cells of the invention are set out below.

Retroviral Transduction of Human T Cells

The following provides a protocol for the production of cells of the invention by transfection with nucleic acids of the invention.

Day −2: Thaw Phoenix Ampho Cells

Late afternoon get Phoenix Ampho cells (retroviral packaging cell line for transduction of human cells) out of −80 and place in culture. Phoenix Ampho cells are grown in DMEM with 10% FCS, 1% L-glut (no antibiotics). Phoenix Ampho cells should never reach confluency. Typically put 2-3×10⁶ Phoenix Ampho cells in each T150 flask in 30 ml of media. On day 0 you should have around 30-40×10⁶ Phoenix Ampho cells.

Day 1: Set Up Phoenix Ampho Cells

Trypsinise Phoenix Ampho cells using TryLE and set up Phoenix Ampho cells at 8×10⁶ cells/flask in 30 ml DMEM with 10% FCS and 1% L-glutamine (no antibiotics) (volume for T150 flask, scale as appropriate). Incubate cells overnight (37° C./5% CO₂).

Day 2: Transfection of Phoenix Ampho Cells

Phoenix Ampho cells should be 50-80% confluent on the day of transfection. The cells should then be transfected by the following method (for a T150 flask, scale as appropriate if using different flasks).
1. For each T150 flask of phoenix cells, place 12 pg of plasmid DNA (i.e. CAR plasmid)+12 μg pCI ampho plasmid into a 15 ml falcon and make up to 1800 μl with OptiMEM (Gibco) mixing gently with a pipette. To another 15 ml falcon add 1680 μl OptiMEM and add 120 μl Fugene 6 transfection reagent (available from stores) ensuring Fugene goes directly into OptiMEM rather than sticking to sides of tube; mix gently with a pipette. Then add the 1800 μl of OptiMEM/fugene mix to the tubes containing the plasmid DNA and mix gently with a pipette. Incubate at room temp for 45 mins. This allows fugene to form complexes with the DNA that have a neutral charge allowing DNA to be transported across the negatively charged Phoenix Ampho cell membrane.
2. Very gently replace the media on the Phoenix Ampno cells to 9 mls fresh DMEM with 10% FCS and glutamine then immediately overlay the DNA/fugene complexes or OptiMEM (for mock controls) onto the cells. Gently mix by north-south and east-west movements of the plate.
3. Incubate cells for 24 hours (37° C./5% CO₂).

Day 2: Activation of T Cells

T cells will not expand in the first 48 hours after activation, so typically activate as many T cells as you need (or more in case of cell death) for your transduction.

Method Using Anti-CD3/CD28 Antibodies:
1. Lymphoprep a fresh leukocyte cone.
2. Count cells and culture at 1×10⁶/ml in T cell media (1% human serum, 10% FCS, P/S, L-glut RPMI). Typically 200 mls per T150 flask.
3. Add IL-2 at 300 U/ml, add OKT3 (anti-CD3) at 30 ng/ml, add anti-CD28 mAB at 30 ng/ml (#MAB342-SP, R&D).
4. Incubate at 37° C./5% CO2 for 48 hours.

Method Using Anti-CD3/CD28 Dynabeads:
1. Lymphoprep a fresh cone. Count cells and assume that 50% of PBMCs are CD3+ T cells.
2. Resuspend cells in a 15 ml falcon at 10×10⁶ CD3+ T cells per ml of 5% human serum, PBS.
3. Add two Dynabeads® Human T-Activator CD3/CD28 per CD3+ cell. Washing dynabeads: vortex vial of beads for 30s. Remove required volume of beads and place in a 15 ml falcon. Add 1 ml of sterile PBS and mix well with a pipette. Place falcon on the dynabead magent—dynabeads will stick to the edge of the falcon. Carefully remove the supernatant without disturbing the beads. Take falcon off the magnet and repeat wash step. Add dynabeads to your T cells in a small volume of PBS.
4. Incubate T cells on a tumbler at room temperature for at least an hour. T cells will bind dynabeads during this step, allowing selection of CD3+ T cells and activation at the same time.
5. Place cells on the dynabead magnet to remove non-bound cells. Count cells and culture at 1×10⁶/ml in T cell media (1% human serum, 10% FCS, P/S, L-glut RPMI) with IL-2 300 U/ml
6. Incubate at 37° C./5% CO2 for 48 hours.

Day 3: Change Phoenix Ampho Media

Phoenix ampho cells will now be producing retrovirus containing your plasmid DNA, so take this into account when handling cells/supernatants. Place an autoclave bag inside your TC hood and place any plastics contaminated with retrovirus (cells/sups) within it. When you are finished, seal the autoclave bag and place into an autoclave tin. Put any liquid waste in a waste pot and seal. Take retrovirus contaminated waste to wash-up ASAP.

Gently replace medium on Phoenix Ampho cells with fresh 21 mls/flask (volume for T150 flask, scale as appropriate) of DMEM+10% FCS+2 mM L-glutamine (no antibiotics). Incubate the cells for a further 24 hours.

Day 4: Transduction of Human T-Cells
1. Add 2 mls of retronectin (30 μg/ml) (#T100B—Takara RetroNectin® Recombinant Human Fibronectin Fragment) to each of the required number of wells of a 6-well plate (non tissue culture-treated) and incubate at room temperature for 3 hours (can also be set up the day before and coated overnight in the fridge). Remember to include wells for mock-transfected controls in the experiment. Culture plates are coated in retronectin to co-localise T cells and virus particles to allow efficient transduction of cells
2. Remove retronectin (it can be re-used until it has run out) and block wells with 2.5 ml of sterile PBS/2% BSA/well for 30 mins. Remove the blocking solution and wash wells twice with 2.5 mls of sterile PBS (keep last PBS wash on well until ready to add virus).
3. Pre-warm some T cell media.
4. Pre-warm centrifuge for spinfection by spinning with empty buckets at 3160 rpm/2000 g for 60 mins@32° C. This can be interrupted when ready to do spinfection.
5. Harvest retrovirus-containing culture supernatant from Phoenix Ampho cells and spin down (1500 rpm for 5 mins). Transfer retrovirus sup to fresh tubes. Some people filter their retrovirus using a 0.45 μm filter, to remove contaminating Phoenix ampho cells, but this could decrease retroviral titre. If necessary, the virus can be snap frozen on dry ice/ethanol slurry and stored at −80° C., but titre is halved with every freeze thawing.
6. Spinfection: Add 2 ml/well of virus supernatant (or mock supernatant) to retronectin-coated wells and spin at 3160 rpm/2000 g for 2 hours @ 32° C.
7. 45 mins before this spin finishes, prepare the T cells due to be transduced. Harvest T cells and count. Resuspend T cells at 1×10⁶ in T cell media+IL2 (100 U/ml) and incubate (37° C./5% CO₂) for 15-20 mins to allow cells to recover from centrifugation.
8. When virus has finished spinning, remove supernatants and wash wells once with PBS (2.5 ml/well).
9. Remove PBS from virus/retronectin coated plate and add T cells due to be transduced (2 ml/well). Ensure cells evenly distributed over plate by rocking north:south and east:west. Spin plates at 1300 rpm for 5 mins.
10. Place plates in incubator (37° C./5% CO₂).

Day 5: Feed Transduced T Cells

Add another 6 ml of T cell media+IL2 (100 IU/ml) to each well of T cells and return to incubator (37° C./5% CO₂).

Determining CAR Transduction Efficiency

The efficiency of methods for transducing cells to produce cells of the invention may be determined using the following procedure.

CAR T cell transduction efficiency is determined 4 days post-spinfection. Take samples from mock and CAR T cell wells and stain as follows:
1. Wash ×1 with FACs buffer (10% FCS, PBS)
2. Stain with CD34-APC (1 μl/sample), CD4-FITC (2 μl/sample) and CD8-PE (1 μl/sample) in 50 μl of FACs buffer
3. Incubate for 20 mins on ice
4. Wash ×1 with FACS buffer
5. Resuspend cells in 200 μl of FACs buffer and analyse by flow cytometry (LSRII).

Sorting Cells of the Invention (Such as CAR T Cells) by CD34 Magnetic-Activated Cell Sorting

CAR-transduced cells (such as T cells) are sorted as follows:
1. Spin down T-cells at 1500 rpm, 5 mins and pour off supernatant.
2. Resuspend cells in 10 ml cold MACS buffer and spin 1500 rpm, 5 mins and pour off supernatant.
3. Resuspend cells in 300 μl cold MACS buffer, add 100 μl FcR blocking agent and 100 μl CD34 microbeads (Miltenyi Biotech 130-046-702). These quantities are suitable for up to 10⁸ cells—if more than that, scale up accordingly.
4. Mix well and incubate for 30 mins in the fridge (2-8° C.).
5. Wash in 50 ml cold MACS buffer and spin 1500 rpm, 5 mins and pour off supernatant.
6. Resuspend cells in 500 μl cold MACS buffer and load cell suspension onto an MS column that has been pre-rinsed with 500 μl cold MACS buffer.
7. Allow cells to drip through by gravity flow and wash columns 3 times with 500 μl cold MACS buffer.
8. Remove columns from the magnet and flush through with 1 ml cold MACS buffer, collecting the cells in a sterile tube.
9. Centrifuge sorted CAR T cells and resuspend in normal T cell media (1% human serum, 10% FBS, P/S, L-glut, 100U/ml IL-2, RPMI 1640) at a concentration of 1×10⁶ CAR T cells per ml.
10. Check purity of CAR T cells by CD34 surface antibody staining the following day.

Protocol to Measure Arginase Activity of Cells of the Invention

EQUIPMENT & MATERIALS
Microcentrifuge
Heatblock
Waterbath
Spectrophotometer
Ice bucket and ice
PROTOCOL
Cell Lysis
Count the number of cells. Use approximately 0.5 × 10 to 1 × 10 per sample
Make up the following lysis buffer per sample:
50 uL 0.1% Triton X-100 + Protease Inhibitor (25x)
Add to the sample and place at 37° C. heatblock for 30 mins. Vortex every 10
mins.
Spin at 13000 rpm for 20 seconds and collect supernatant
In the meantime, warm water bath/heatblock to 56° C./
Enzyme Activation
Add 50 uL ofTRIS-HCl 25 mM, followed by 10 uL MnCL2 10 mM (Total 60 uL)
to 50 uL supernatant.
ADD EACH COMPONENT SEPARATELY otherwise background
will be high
Heat samples to 56° C. for 10 mins
Put samples on ice for 5-10 mins
Reset heatblock to 37° C.
Hydrolysis of Arginine
Add 100 uL L-arginine 0.5M to each sample. Place on heatblock for
120 mins.
L-arginine: molecular weight = 174.2 → in 10 ml require 0.871 g
(dissolved in water)
Standard Curve
Make 1:2 dilutions down to 0.78 ug/ml/1:1024 of urea (10 ug/ml)
Pipette 50 uL of each standard into an Eppendorf tube and add 50 uL TRIS-
HCl + 10 uL MnCl2 + 100 uL L-Arginine
NB: Blanks: 50 uL 0.1% Triton X-100, 50 uL H2O
DO NOT PUT THESE SAMPLES ON HEAT BLOCK UNTIL COLOUR-READOUT
STAGE
Blocking the Reaction
Once the sample hydrolysis is complete, add 800 uL of the following stop
solution per sample and standards (ratio below) [1:3:7]
H2SO4 100 uL
H3PO4 300 uL
Water 700 uL
Colour Readout for Urea
Add 40 uL of 9% di-alpha-isonitrosopropiophenone (dissolved in 100%
EtOH)
Heat for 30 mins at 100° C.
NB: pierce the top of the eppendorfs with a needle before boiling
Continue the reaction until the standards have turned purple
Put samples on ice for 10 mins in the dark
Centrifuge samples for 5 mins at 13000 rpm
Plate 200 uL of each sample supernatant into a flat 96 well plate in duplicate.
Read at 540 nm in a cell plate reader

Protocol to Measure Cell Proliferation

EQUIPMENT & MATERIALS
Incubator
Cytoflex
PROTOCOL
Unsorted T cells, 7 days post transduction were plated in 2 ml of either normal Arg
conditions (RPMI 10% FBS), Arg− condition (SILAC RPMI 10% Dyalysed FBS) or
Arg+ condition (RPMI 10% FBS + 100 ng/ml L-Arg) in 6 well plates, at a density of
1 × 10⁶ cells/well. All media supplemented with IL-2 (100 U/ml).
Plates were incubated at 37° C., 5% CO2 for 7 days and the cells were subsequently
harvested and centrifuged at 1500 rpm for 5 minutes.
Transduced T cells were washed with FACS buffer and stained with anti-CD34-APC.
Samples were then resuspended in FACS buffer for reading.
1 μl of PI (1 mg/ml) was added to each sample immediately prior to acquisition, in
order to stain dead/dying cells. Samples were acquired for set time of 60 seconds
using Cytoflex and analysed using Flowjo software.
The proliferation of CAR T cells (CD34+ cells) was normalized to the transduction
efficiency of each construct and the resulting cell count expressed as fold change
compared to the unmodified GD2 CAR in the relative culturing medium.

CAR-T Cell Proliferation Assay

EQUIPMENT & MATERIALS
Anti CD3 antibody
Anti CD28 antibody
Thymidine
Complete medium: RPMI 10% FCS + bME + IL2 (100 U/ml)
SILAC RPMI 10% Dyalysed FCS + bME + IL2 (100 U/ml)
PROTOCOL
Pre-coat the night before a 96 well flat bottom plate with anti CD3
antibody (3 μg/ml in 50 ul RPMI-10% + bME)
MACS-sorted CAR T-cells from healthy donors are counted and plated
at a concentration of 100,000 cells/well in 75% Arginine Free
medium + 25% complete medium
Add anti CD28 at 2 μg/ml
After 4 days supplement with 50 μl of complete medium.
24 h after, add thymidine: 1 μCi/well is equal to 1 μl of thymidine/well.
Add 1 μl of thymidine + 24 μl complete medium and add to the culture
for less than 16 hours
Harvest the plate and read on TopCount to detect thymidine
incorporation (cpm).
The proliferation of the enzyme-containing CARs was determined
using the following equation:
$\frac{cpm of enzyme - containing CAR T cells}{cpm of normal GD 2 CAR T cells} \times 100$

SEQUENCE INFORMATION
SEQ ID NO: 1
MSAKSRTIGIIGAPFSKGQPRGGVEEGPTVLRKAGLLEKLKEQECDVKDYGDLPFADIPNDSPFQIVKNPRSVGKASE
QLAGKVAEVKKNGRISLVLGGDHSLAIGSISGHARVHPDLGVIWVDAHTDINTPLTTTSGNLHGQPVSFLLKELKGKI
PDVPGFSWVTPCISAKDIVYIGLRDVDPGEHYILKTLGIKYFSMTEVDRLGIGKVMEETLSYLLGRKKRPIHLSFDVDG
LDPSFTPATGTPVVGGLTYREGLYITEEIYKTGLLSGLDIMEVNPSLGKTPEEVTRTVNTAVAITLACFGLAREGNHKPI
DYLNPPK
SEQ ID NO: 2
ATGAGCGCCAAGTCCAGAACCATAGGGATTATTGGAGCTCCTTTCTCAAAGGGACAGC
CACGAGGAGGGGTGGAAGAAGGCCCTACAGTATTGAGAAAGGCTGGTCTGCTTGAGA
AACTTAAAGAACAAGAGTGTGATGTGAAGGATTATGGGGACCTGCCCTTTGCTGACATC
CCTAATGACAGTCCCTTTCAAATTGTGAAGAATCCAAGGTCTGTGGGAAAAGCAAGCGA
GCAGCTGGCTGGCAAGGTGGCAGAAGTCAAGAAGAACGGAAGAATCAGCCTGGTGCT
GGGCGGAGACCACAGTTTGGCAATTGGAAGCATCTCTGGCCATGCCAGGGTCCACCCT
GATCTTGGAGTCATCTGGGTGGATGCTCACACTGATATCAACACTCCACTGACAACCAC
AAGTGGAAACTTGCATGGACAACCTGTATCTTTCCTCCTGAAGGAACTAAAAGGAAAGA
TTCCCGATGTGCCAGGATTCTCCTGGGTGACTCCCTGTATATCTGCCAAGGATATTGTG
TATATTGGCTTGAGA
GACGTGGACCCTGGGGAACACTACATTTTGAAAACTCTAGGCATTAAATACTTTTCAAT
GACTGAAGTGGACAGACTAGGAATTGGCAAGGTGATGGAAGAAACACTCAGCTATCTA
CTAGGAAGAAAGAAAAGGCCAATTCATCTAAGTTTTGATGTTGACGGACTGGACCCATC
TTTCACACCAGCTACTGGCACACCAGTCGTGGGAGGTCTGACATACAGAGAAGGTCTC
TACATCACAGAAGAAATCTACAAAACAGGGCTACTCTCAGGATTAGATATAATGGAAGT
GAACCCATCCCTGGGGAAGACACCAGAAGAAGTAACTCGAACAGTGAACACAGCAGTT
GCAATAACCTTGGCTTGTTTCGGACTTGCTCGGGAGGGTAATCACAAGCCTATTGACTA
CCTTAACCCACCTAAG
SEQ ID NO: 3
MSLRGSLSRLLQTRVHSILKKSVHSVAVIGAPFSQGQKRKGVEHGPAAIREAGLMKRLSSLGCHLKDFGDLSFTPVP
KDDLYNNLIVNPRSVGLANQELAEVVSRAVSDGYSCVTLGGDHSLAIGTISGHARHCPDLCVVWVDAHADINTPLT
TSSGNLHGQPVSFLLRELQDKVPQLPGFSWIKPCISSASIVYIGLRDVDPPEHFILKNYDIQYFSMRDIDRLGIQKVME
RTFDLLIGKRQRPIHLSFDIDAFDPTLAPATGTPVVGGLTYREGMYIAEEIHNTGLLSALDLVEVNPQLATSEEEAKTT
ANLAVDVIASSFGQTREGGHIVYDQLPTPSSPDESENQARVRI
SEQ ID NO: 4
ATGTCTTTAAGAGGCTCTTTATCTCGTCTGCTGCAGACTCGTGTGCACAGCATTTTAAAA
AAGAGCGTGCACAGCGTGGCCGTCATTGGAGCCCCCTTCAGCCAAGGCCAGAAGAGA
AAGGGCGTCGAACACGGACCCGCCGCCATCAGAGAAGCTGGTTTAATGAAGAGACTGA
GCTCTTTAGGCTGCCATTTAAAAGACTTCGGAGATTTGTCTTTTACCCCCGTCCCCAAG
GACGATTTATACAATAATTTAATCGTGAACCCCAGATCCGTGGGACTGGCTAACCAAGA
ACTGGCCGAGGTCGTGAGCAGAGCCGTGTCCGACGGCTACTCTTGTGTGACTTTAGGC
GGCGATCACTCTTTAGCCATTGGCACAATCTCCGGACACGCTAGGCACTGCCCCGATT
TATGCGTGGTGTGGGTGGACGCTCACGCCGACATCAATACCCCTCTGACCACCAGCAG
CGGCAATTTACACGGACAGCCCGTCAGCTTTTTACTGAGGGAGCTGCAAGATAAGGTG
CCTCAGCTGCCCGGCTTCAGCTGGATCAAGCCTTGTATCAGCAGCGCTTCCATCGTGT
ACATTGGTTTAAGAGACGTGGACCCTCCCGAACACTTCATCCTCAAGAACTACGACATT
CAGTACTTCAGCATGAGGGATATCGATCGTCTCGGAATCCAGAAGGTGATGGAAAGGA
CCTTCGATTTACTCATCGGAAAGAGGCAGAGGCCTATCCATTTATCCTTCGACATCGAC
GCCTTCGATCCTACACTGGCCCCCGCTACTGGTACACCCGTTGTGGGCGGTTTAACCT
ATAGGGAGGGCATGTACATCGCCGAAGAGATCCACAACACCGGTTTACTGAGCGCTCT
GGATTTAGTGGAGGTGAATCCTCAGCTGGCCACCTCCGAGGAGGAGGCCAAAACCAC
CGCCAATCTGGCCGTGGACGTGATCGCCAGCTCCTTCGGCCAGACCAGAGAGGGCGG
CCACATTGTGTACGACCAGCTGCCCACACCCAGCTCCCCCGATGAGTCCGAAAACCAA
GCTCGTGTCAGAATCTGATAATAACCCAAGCTT
- Amino acid seguence of exemplary GD2 target binding moiety
Sequence ID NO: 5
DILLTQTPLSLPVSLGDQASISCRSSQSLVHRNGNTYLHWYLQKPGQSPKLLIHKVSNRFSGVPDRFS
GSGSGTDFTLKISRVEAEDLGVYFCSQSTHVPPLTFGAGTKLELKRADAAPTVSIFPGSGGGGSGGE
VKLQQSGPSLVEPGASVMISCKASGSSFTGYNMNWVRQNIGKSLEWIGAIDPYYGGTSYNQKFKGR
ATLTVDKSSSTAYMHLKSLTSEDSAVYYCVSGMEYWGQGTSVTVSSAKTTPPSVYGRVTVSS
- Amino acid seguence of exemplary CD33 target binding moiety
Sequence ID NO: 6
GSNIMLTQSPSSLAVSAGEKVTMSCKSSQSVFFSSSQKNYLAWYQQIPGQSPKLLIYWASTRESGVP
DRFTGSGSGTDFTLTISSVQSEDLAIYYCHQYLSSRTFGGGTKLEIKRGGGGSGGGGSSGGGSQVQ
LQQPGAEVVKPGASVKMSCKASGYTFTSYYIHWIKQTPGQGLEWVGVIYPGNDDISYNQKFKGKATL
TADKSSTTAYMQLSSLTSEDSAVYYCAREVRLRYFDVWGAGTTVTVSS
- Amino acid seguence of exemplary mesothelin target binding moiety
Sequence ID NO: 7
MQVQLQQSGPELEKPGASVKISCKASGYSFTGYTMNWVKQSHGKSLEWIGLITPYNGASSYNQKFR
GKATLTVDKSSSTAYMDLLSLTSEDSAVYFCARGGYDGRGFDYWGQGTTVTVSSGVGGSGGGGSG
GGGSDIELTQSPAIMSASPGEKVTMTCSASSSVSYMHWYQQKSGTSPKRWIYDTSKLASGVPGRFS
GSGSGNSYSLTISSVEAEDDATYYCQQWSGYPLTFGAGTKLEIK
- Amino acid seguence of exemplary EGFRVIII target binding moiety
Sequence ID NO: 8
QVQLQQSGGGLVKPGASLKLSCVTSGFTFRKFGMSWVRQTSDKRLEWVASISTGGYNTYYSDNVK
GRFTISRENAKNTLYLQMSSLKSEDTALYYCTRGYSSTSYAMDYWGQGTTVTVSSSGGGSGGGGS
GGGGSDIELTQSPASLSVATGEKVTIRCMTSTDIDDDMNWYQQKPGEPPKFLISEGNTLRPGVPSRF
SSSGTGTDFVFTIENTLSEDVGDY
- DNA encoding an alternative EGFRvlll target binding moiety
(EGFRvIII scFv seguence derived from MR1 antibody)
Sequence ID No: 9
Atggactggatttggcgcatccttttccttgtcggcgctgctaccggcgcgcattctcaggtacaact
ccagcagtctgggggaggcttagtgaagcctggagcgtctctgaaactctcctgtgtaacctctggat
tcactttcagaaaatttggcatgtcttgggttcgccagactagtgacaagaggctggaatgggtcgca
tccattagtactggcggttataacacgtactattcagacaatgtaaagggccgattcaccatctccag
agagaatgccaagaacaccctgtacctgcaaatgagtagtctgaagtctgaggacacggccttgtatt
actgtacaagaggctattctagtacctcttatgctatggactactggggccaagggaccacggtcacc
gtctcctcaagtggaggcggttcaggcggaggtggctctggcggtggcggatcggacatcgagctcac
tcagtctccagcatccctgtccgtggctacaggagaaaaagtcactatcagatgcatgaccagcactg
atattgatgatgatatgaactggtaccagcagaagccaggggaaccccctaagttccttatttcagaa
ggcaatactcttcggcegggagtcccatcccgattttccagcagtggcactggcacagattttgtttt
tacaattgaaaacacactctcggaagatgttggagattactactgtttgcaaagctttaacgtgcctc
ttacattcggtgatggcaccaagcttgaaaaagctcta
SEQ ID NO: 10, alternative EGFRvIII binding moiety encoded by
SEQ ID NO: 9
MDWIWRILFLVGAATGAHSQVQLQQSGGGLVKPGASLKLSCVTSGFTFRKFGMSWVRQTSDKRLEWVASISTGG
YNTYYSDNVKGRFTISRENAKNTLYLQMSSLKSEDTALYYCTRGYSSTSYAMDYWGQGTTVTVSSSGGGSGGGGS
GGGGSDIELTQSPASLSVATGEKVTIRCMTSTDIDDDMNWYQQKPGEPPKFLISEGNTLRPGVPSRFSSSGTGTDF
VFTIENTLSEDVGDYYCLQSFNVPLTFGDGTKLEKAL
- Amino acid seguence of exemplary 4-1BB intracellular signalling region
Sequence ID NO: 11
KRGRKKLLYIFKQPFMRPVQTTQEEDGCSCRFPEEEEGGCEL
- Amino acid seguence of exemplary OX-40 intracellular signalling region
Sequence ID NO: 12
RDQRLPPDAHKPPGGGSFRTPIQEEQADAHSTLAKI
- Amino acid seguence of exemplary CD28 intracellular signalling region
with transmembrane domain
Sequence ID NO: 13
IEVMYPPPYLDNEKSNGTIIHVKGKHLCPSPLFPGPSKPFWVLVVVGGVLACYSLLVTVAFIIFWVRSK
RSRLLHSDYMNMTPRRPGPTRKHYQPYAPPRDFAAYRS
- Amino acid seguence of exemplary ICOS intracellular signalling region
Sequence ID NO: 14
CWLTKKKYS SSVHDPNGEY MFMRAVNTAK KSRLTDVTL
(the cytoplasmic portion of ICOS, comprising residues 162-199 of the
full-length protein.
The motif YMFM (residues 180-183 of the full-length protein) is of
particular relevance, and should be retained in an ICOS intracellular
signally region suitable for use in a protein of the invention)
- Amino acid seguence of exemplary CD3 ζ intracellular signalling region
Sequence ID NO: 15
RVKFSRSADAPAYKQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
- Alternative CD3z
Sequence ID NO: 16
AGAGTGAAGTTCAGCAGGAGCGCAGACGCCCCCGCGTACCAGCAGGGCCAGAACCAGCTCTAT
AACGAGCTCAATCTAGGACGAAGAGAGGAGTACGATGTTTTGGACAAGAGACGTGGCCGGGAC
CCTGAGATGGGGGGAAAGCCGCAGAGAAGGAAGAACCCTCAGGAAGGCCTGTACAATGAACTG
CAGAAAGATAAGATGGCGGAGGCCTACAGTGAGATTGGGATGAAAGGCGAGCGCCGGAGGGG
CAAGGGGCACGATGGCCTTTACCAGGGTCTCAGTACAGCCACCAAGGACACCTACGACGCCCTT
CACATGCAGGCCCTGCCCCCTCGCTAA
SEQ ID NO 17
Amino acid seguence of anti-GD2 CAR incorporating arginase type I
MPRGWTALCLLSLLPSGFMSLDNNGTATPELPTQGTFSNVSTNVSYQETTTPSTLGSTSL
HPVSQHGNEATTNITETTVKFTSTSVITSVYGNTNSSVQSQTSVISTVFTTPANVSTPET
TLKPSLSPGNVSDLSTTSTSLATSPTKPYTSSSPILSDIKAEIKCSGIREVKLTQGICLE
QNKTSSCAEFKKDRGEGLARVLCGEEQADADAGAQVCSLLLAQSEVRPQCLLLVLANRTE
ISSKLQLMKKHQSDLKKLGILDFTEQDVASHQSYSQKTLIALVTSGALLAVLGITGYFLM
NRRSWSPTGERLELEPVDRVKQTLNFDLLKLAGDVESNPGPGNMALPVTALLLPLALLLH
AARPDILLTQTPLSLPVSLGDQASISCRSSQSLVHRNGNTYLHWYLQKPGQSPKLLIHKV
SNRFSGVPDRFSGSGSGTDFTLKISRVEAEDLGVYFCSQSTHVPPLTFGAGTKLELKRAD
AAPTVSIFPGSGGGGSGGEVKLQQSGPSLVEPGASVMISCKASGSSFTGYNMNWVRQNIG
KSLEWIGAIDPYYGGTSYNQKFKGRATLTVDKSSSTAYMHLKSLTSEDSAVYYCVSGMEY
WGQGTSVTVSSAKTTPPSVYGRVTVSSAEPKSCDKTHTCPPCPAPELLGGPSVFLFPPKP
KDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSV
MHEALHNHYTQKSLSLSPGKKDPKTTTPAPRPPTPAPTIASQPLSLRPEACRPAAGGAVH
TRGLDFACDIYIWAPLAGTCGVLLLSLVITLYCKRGRKKLLYIFKQPFMRPVQTTQEEDG
CSCRFPEEEEGGCELRVKFSRSADAPAYKQGQNQLYNELNLGRREEYDVLDKRRGRDPEM
GGKPRRKNPQEGLYNELQKDKMAEAYSEIGMKGERRRGKGHDGLYQGLSTATKDTYDALH
MQALPPRGSGATNFSLLKQAGDVEENPGPMSAKSRTIGIIGAPFSKGQPRGGVEEGPTVLRKAGLLEKLKEQECDV
KDYGDLPFADIPNDSPFQIVKNPRSVGKASEQLAGKVAEVKKNGRISLVLGGDHSLAIGSISGHARVHPDLGVIWVD
AHTDINTPLTTTSGNLHGQPVSFLLKELKGKIPDVPGFSWVTPCISAKDIVYIGLRDVDPGEHYILKTLGIKYFSMTEV
DRLGIGKVMEETLSYLLGRKKRPIHLSFDVDGLDPSFTPATGTPVVGGLTYREGLYITEEIYKTGLLSGLDIMEVNPSL
GKTPEEVTRTVNTAVAITLACFGLAREGNHKPIDYLNPPK
SEQ ID NO 18
DNA seguence encoding the protein of SEQ ID NO: 17
ctcgagagctttggcgtaatcatggtcatagctgtttcctgtgtgaaattgttatccgctcacaattccacacaacatacga
gccggaagcataaagtgtaaagcctggggtgcctaatgagtgagctaactcacattaattgcgttgcgctcactgccc
gctttccagtcgggaaacctgtcgtgccagctgcattaatgaatcggccaacgcgcggggagaggcggtttgcgtatt
gggcgctcttccgcttcctcgctcactgactcgctgcgctcggtcgttcggctgcggcgagcggtatcagctcactcaa
aggcggtaatacggttatccacagaatcaggggataacgcaggaaagaacatgtgagcaaaaggccagcaaaagg
ccaggaaccgtaaaaaggccgcgttgctggcgtttttccataggctccgcccccctgacgagcatcacaaaaatcga
cgctcaagtcagaggtggcgaaacccgacaggactataaagataccaggcgtttccccctggaagctccctcgtgc
gctctcctgttccgaccctgccgcttaccggatacctgtccgcctttctcccttcgggaagcgtggcgctttctcaatgct
cacgctgtaggtatctcagttcggtgtaggtcgttcgctccaagctgggctgtgtgcacgaaccccccgttcagcccga
ccgctgcgccttatccggtaactatcgtcttgagtccaacccggtaagacacgacttatcgccactggcagcagccac
tggtaacaggattagcagagcgaggtatgtaggcggtgctacagagttcttgaagtggtggcctaactacggctacac
tagaaggacagtatttggtatctgcgctctgctgaagccagttaccttcggaaaaagagttggtagctcttgatccggca
aacaaaccaccgctggtagcggtggtttttttgtttgcaagcagcagattacgcgcagaaaaaaaggatctcaagaag
atcctttgatcttttctacggggtctgacgctcagtggaacgaaaactcacgttaagggattttggtcatgagattatcaaa
aaggatcttcacctagatccttttaaattaaaaatgaagttttaaatcaatctaaagtatatatgagtaaacttggtctgaca
gttaccaatgcttaatcagtgaggcacctatctcagcgatctgtctatttcgttcatccatagttgcctgactccccgtcgtg
tagataactacgatacgggagggcttaccatctggccccagtgctgcaatgataccgcgagacccacgctcaccggc
tccagatttatcagcaataaaccagccagccggaagggccgagcgcagaagtggtcctgcaactttatccgcctccat
ccagtctattaattgttgccgggaagctagagtaagtagttcgccagttaatagtttgcgcaacgttgttgccattgctgct
ggcatcgtggtgtcacgctcgtcgtttggtatggcttcattcagctccggttcccaacgatcaaggcgagttacatgatc
ccccatgttgtgcaaaaaagcggttagctccttcggtcctccgatcgttgtcagaagtaagttggccgcagtgttatcact
catggttatggcagcactgcataattctcttactgtcatgccatccgtaagatgcttttctgtgactggtgagtactcaacc
aagtcattctgagaatagtgtatgcggcgaccgagttgctcttgcccggcgtcaatacgggataataccgcgccacat
agcagaactttaaaagtgctcatcattggaaaacgttcttcggggcgaaaactctcaaggatcttaccgctgttgagatc
cagttcgatgtaacccactcgtgcacccaactgatcttcagcatcttttactttcaccagcgtttctgggtgagcaaaaac
aggaaggcaaaatgccgcaaaaaagggaataagggcgacacggaaatgttgaatactcatactcttcctttttcaatat
tattgaagcatttatcagggttattgtctcatgagcggatacatatttgaatgtatttagaaaaataaacaaataggggttcc
gcgcacatttccccgaaaagtgccacctgacgtctaagaaaccattattatcatgacattaacctataaaaataggcgta
tcacgaggccctttcgtcttcaagctgcctcgcgcgtttcggtgatgacggtgaaaacctctgacacatgcagctcccg
gagacggtcacagcttgtctgtaagcggatgccgggagcagacaagcccgtcagggcgcgtcagcgggtgttggc
gggtgtcggggcgcagccatgacccagtcacgtagcgatagttactatgcggcatcagagcagattgtactgagagt
gcaccatatgcggtgtgaaataccgcacagatgcgtaaggagaaaataccgcatcaggcgccattcgccattcaggc
tgcgcaactgttgggaagggcgatcggtgcgggcctcttcgctattacgccagctggcgaaagggggatgtgctgca
aggcgattaagttgggtaacgccagggttttcccagtcacgacgttgtaaaacgacggccagtgaattagtactctagc
ttaagtaacgccattttgcaaggcatggaaaatacataactgagaatagagaagttcagatcaaggttaggaacagag
agacagcagaatatgggccaaacaggatatctgtggtaagcagttcctgccccggctcagggccaagaacagttgg
aacagcagaatatgggccaaacaggatatctgtggtaagcagttcctgccccggctcagggccaagaacagatggt
ccccagatgcggtcccgccctcagcagtttctagagaaccatcagatgtttccagggtgccccaaggacctgaaatg
accctgtgccttatttgaactaaccaatcagttcgcttctcgcttctgttcgcgcgcttctgctccccgagctcaataaaag
agcccacaacccctcactcggcgcgccagtcctccgatagactgcgtcgcccgggtacccgtattcccaataaagcc
tcttgctgtttgcatccgaatcgtggactcgctgatccttgggagggtctcctcagattgattgactgcccacctcggggg
tctttcatttggaggttccaccgagatttggagacccctgcccagggaccaccgacccccccgccgggaggtaagct
ggccagcggtcgtttcgtgtctgtctctgtctttgtgcgtgtttgtgccggcatctaatgtttgcgcctgcgtctgtactagtt
ggctaactagatctgtatctggcggtcccgcggaagaactgacgagttcgtattcccggccgcagcccctgggagac
gtcccagcggcctcgggggcccgttttgtggcccattctgtatcagttaacctacccgagtcggactttttggagctccg
ccactgtccgaggggtacgtggctttgttgggggacgagagacagagacacttcccgcccccgtctgaatttttgcttt
cggttttacgccgaaaccgcgccgcgcgtcttgtctgctgcagcatcgttctgtgttgtctctgtctgactgtgtttctgtatt
tgtctgaaaattagctcgacaaagttaagtaatagtccctctctccaagctcacttacaggcggccgaattcgccgccg
ccatgcctcgcggctggacagccctgtgcctgctgtctctgctgccatccggcttcatgagcctggataataacggcacagccac
cccagagctgcctacacagggcaccttcagcaatgtgtccacaaacgtgagctatcaggagaccacaaccccttctaccctgg
gatccacaagcctgcaccccgtgtctcagcacggcaacgaagccaccaccaacatcaccgagaccacagtgaagtttacctc
cacctctgtgattacctctgtgtacggaaatacaaactccagcgtgcagtctcagacatctgtgatctccacagtgtttacaacac
ctgccaatgtgtccaccccagagacaaccctgaagcccagcctgtctcctggaaatgtgtccgatctgtctaccacctccaccagcc
tggccacctctcccaccaagccctatacctcctcttctcccatcctgagcgatatcaaagccgagatcaaatgcagcgggattcgg
gaagtgaaactgacacagggcatctgcctggaacagaataagacatccagctgcgccgagtttaagaaagatagaggagag
ggactggccagggtgctgtgtggcgaagagcaggccgacgccgatgccggcgcccaggtgtgttccctgctgctggcccagtc
tgaggtgcgcccccagtgcctgctgctggtgctggccaatcggacagaaattagcagcaagctgcagctgatgaaaaaacacc
agagcgatctgaaaaagctgggcatcctggactttaccgagcaggacgtggcctctcaccagagctacagccagaaaacact
gatcgccctggtgaccagcggagccctgctggccgtgctgggcatcaccggatatttcctgatgaataggcgcagctggagccc
caccggcgaacggctggagctggagcctgtcgaccgagtgaagcagaccctgaactttgatctgctgaagctggccggcgac
gtggagtccaaccccgggccagggaatATGGCCTTACCAGTGACCGCCTTGCTCCTGCCGCTGGC
CTTGCTGCTCCACGCCGCCAGGCCGGATATTCTGCTCACACAGACCCCACTCTCCCTG
CCCGTGTCACTCGGGGATCAGGCTAGCATTTCTTGCCGCTCATCTCAGTCTCTGGTCC
ACCGGAATGGGAACACATACCTCCATTGGTACCTCCAGAAACCTGGACAGAGCCCTAA
ACTGCTCATCCACAAAGTCTCAAATCGGTTCTCCGGCGTGCCCGATCGCTTTAGCGGAT
CCGGATCTGGGACCGACTTCACACTGAAAATCTCACGAGTGGAGGCTGAGGATCTCGG
CGTCTACTTCTGTAGTCAGAGTACCCACGTCCCACCCCTCACCTTTGGCGCTGGAACAA
AACTGGAGCTGAAACGAGCCGATGCTGCTCCTACCGTGTCCATCTTTCCTGGCTCCGG
GGGAGGCGGGAGCGGAGGCGAAGTGAAACTCCAGCAGTCTGGCCCTTCTCTCGTGGA
ACCTGGCGCTTCTGTGATGATCTCCTGTAAGGCCTCTGGATCTTCCTTTACCGGCTACA
ACATGAACTGGGTCCGGCAGAACATTGGCAAATCCCTGGAATGGATTGGCGCCATCGA
TCCTTACTACGGCGGCACATCATACAATCAGAAATTCAAGGGGCGAGCAACACTCACTG
TCGACAAATCTTCATCCACCGCCTACATGCACCTGAAATCTCTCACATCCGAGGATAGT
GCTGTCTACTACTGTGTCTCTGGCATGGAATACTGGGGACAGGGAACTTCTGTCACCG
TGTCTAGTGCCAAAACCACACCTCCCTCCGTGTACGGACGAGTCACTGTCTCATCTGCT
GAACCAAAATCCTGTGACAAAACACACACATGCCCACCTTGTCCTGCCCCTGAACTGCT
CGGCGGACCTTCCGTCTTTCTGTTTCCCCCCAAACCCAAGGATACACTCATGATTTCTA
GGACCCCCGAAGTCACTTGTGTCGTGGTCGATGTGTCTCACGAGGATCCTGAAGTGAA
ATTCAACTGGTACGTGGACGGAGTCGAGGTCCACAATGCCAAAACAAAACCCCGGGAG
GAACAGTACAATAGCACCTACCGAGTCGTGTCCGTGCTCACCGTCCTCCATCAGGATT
GGCTGAACGGCAAAGAGTACAAGTGTAAAGTGAGTAACAAGGCTCTCCCCGCTCCTAT
TGAAAAAACCATCTCAAAAGCAAAAGGCCAGCCTAGGGAGCCTCAGGTCTACACACTG
CCACCCTCACGGGACGAACTCACCAAAAATCAGGTGTCCCTCACTTGCCTGGTGAAAG
GCTTCTACCCTTCCGATATCGCTGTGGAATGGGAGTCAAATGGGCAGCCCGAAAACAA
CTACAAAACAACCCCCCCTGTGCTCGATTCCGATGGCTCTTTTTTCCTGTACTCCAAACT
CACCGTGGACAAATCACGCTGGCAGCAGGGGAATGTCTTTTCTTGCTCCGTGATGCAC
GAGGCCCTCCACAATCATTACACCCAGAAATCCCTCTCACTCTCACCCGGCAAAAAGGA
CCCTAAAACCACGACGCCAGCACCGCGACCACCAACACCGGCGCCAACCATCGCATC
GCAGCCCCTGTCCCTGCGCCCAGAGGCGTGCCGACCAGCGGCGGGGGGCGCAGTGC
ACACGAGGGGGCTGGACTTCGCCTGTGATATCTACATCTGGGCGCCCTTGGCCGGGA
CTTGTGGGGTCCTTCTCCTGTCACTGGTTATCACCCTTTACTGCAAACGGGGCAGAAAG
AAACTCCTGTATATATTCAAACAACCATTTATGAGACCAGTACAAACTACTCAAGAGGAA
GATGGCTGTAGCTGCCGATTTCCAGAAGAAGAAGAAGGAGGATGTGAACTGAGAGTGA
AGTTCAGCAGGAGCGCAGACGCCCCCGCGTACAAGCAGGGCCAGAACCAGCTCTATA
ACGAGCTCAATCTAGGACGAAGAGAGGAGTACGATGTTTTGGACAAGAGACGTGGCCG
GGACCCTGAGATGGGGGGAAAGCCGAGAAGGAAGAACCCTCAGGAAGGCCTGTACAA
TGAACTGCAGAAAGATAAGATGGCGGAGGCCTACAGTGAGATTGGGATGAAAGGCGAG
CGCCGGAGGGGCAAGGGGCACGATGGCCTTTACCAGGGTCTCAGTACAGCCACCAAG
GACACCTACGACGCCCTTCACATGCAGGCCCTGCCCCCTCGCggcagcggcgccaccaacttca
gcctgctgaagcaggccggcgacgtggaggaaaaccctggccccATGAGCGCCAAGTCCAGAACCATAGG
GATTATTGGAGCTCCTTTCTCAAAGGGACAGCCACGAGGAGGGGTGGAAGAAGGCCCT
ACAGTATTGAGAAAGGCTGGTCTGCTTGAGAAACTTAAAGAACAAGAGTGTGATGTGAA
GGATTATGGGGACCTGCCCTTTGCTGACATCCCTAATGACAGTCCCTTTCAAATTGTGA
AGAATCCAAGGTCTGTGGGAAAAGCAAGCGAGCAGCTGGCTGGCAAGGTGGCAGAAG
TCAAGAAGAACGGAAGAATCAGCCTGGTGCTGGGCGGAGACCACAGTTTGGCAATTGG
AAGCATCTCTGGCCATGCCAGGGTCCACCCTGATCTTGGAGTCATCTGGGTGGATGCT
CACACTGATATCAACACTCCACTGACAACCACAAGTGGAAACTTGCATGGACAACCTGT
ATCTTTCCTCCTGAAGGAACTAAAAGGAAAGATTCCCGATGTGCCAGGATTCTCCTGGG
TGACTCCCTGTATATCTGCCAAGGATATTGTGTATATTGGCTTGAGA
GACGTGGACCCTGGGGAACACTACATTTTGAAAACTCTAGGCATTAAATACTTTTCAAT
GACTGAAGTGGACAGACTAGGAATTGGCAAGGTGATGGAAGAAACACTCAGCTATCTA
CTAGGAAGAAAGAAAAGGCCAATTCATCTAAGTTTTGATGTTGACGGACTGGACCCATC
TTTCACACCAGCTACTGGCACACCAGTCGTGGGAGGTCTGACATACAGAGAAGGTCTC
TACATCACAGAAGAAATCTACAAAACAGGGCTACTCTCAGGATTAGATATAATGGAAGT
GAACCCATCCCTGGGGAAGACACCAGAAGAAGTAACTCGAACAGTGAACACAGCAGTT
GCAATAACCTTGGCTTGTTTCGGACTTGCTCGGGAGGGTAATCACAAGCCTATTGACTA
CCTTAACCCACCTAAGTAATAATAAaagcttaacacgagccatagatagaataaaagattttatttagtctc
cagaaaaaggggggaatgaaagaccccacctgtaggtttggcaagctagcttaagtaacgccattttgcaaggcatg
gaaaatacataactgagaatagagaagttcagatcaaggttaggaacagagagacagcagaatatgggccaaacag
gatatctgtggtaagcagttcctgccccggctcagggccaagaacagttggaacagcagaatatgggccaaacagg
atatctgtggtaagcagttcctgccccggctcagggccaagaacagatggtccccagatgcggtcccgccctcagca
gtttctagagaaccatcagatgtttccagggtgccccaaggacctgaaatgaccctgtgccttatttgaactaaccaatc
agttcgcttctcgcttctgttcgcgcgcttctgctccccgagctcaataaaagagcccacaacccctcactcggcgcgc
cagtcctccgatagactgcgtcgcccgggtacccgtgttctcaataaaccctcttgcagttgcatccgactcgtggtctc
gctgttccttgggagggtctcctctgagtgattgactgcccacctcgggggtctttcatt
SEQ ID NO 19
Amino acid seguence of anti-GD2 CAR incorporating arginase type II
MPRGWTALCLLSLLPSGFMSLDNNGTATPELPTQGTFSNVSTNVSYQETTTPSTLGSTSLHPVSQHGNEATTNITE
TTVKFTSTSVITSVYGNTNSSVQSQTSVISTVFTTPANVSTPETTLKPSLSPGNVSDLSTTSTSLATSPTKPYTSSSPILSD
IKAEIKCSGIREVKLTQGICLEQNKTSSCAEFKKDRGEGLARVLCGEEQADADAGAQVCSLLLAQSEVRPQCLLLVLA
NRTEISSKLQLMKKHQSDLKKLGILDFTEQDVASHQSYSQKTLIALVTSGALLAVLGITGYFLMNRRSWSPTGERLEL
EPVDRVKQTLNFDLLKLAGDVESNPGPGNMALPVTALLLPLALLLHAARPDILLTQTPLSLPVSLGDQASISCRSSQS
LVHRNGNTYLHWYLQKPGQSPKLLIHKVSNRFSGVPDRFSGSGSGTDFTLKISRVEAEDLGVYFCSQSTHVPPLTFG
AGTKLELKRADAAPTVSIFPGSGGGGSGGEVKLQQSGPSLVEPGASVMISCKASGSSFTGYNMNWVRQNIGKSLE
WIGAIDPYYGGTSYNQKFKGRATLTVDKSSSTAYMHLKSLTSEDSAVYYCVSGMEYWGQGTSVTVSSAKTTPPSVY
GRVTVSSAEPKSCDKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHN
HYTQKSLSLSPGKKDPKTTTPAPRPPTPAPTIASQPLSLRPEACRPAAGGAVHTRGLDFACDIYIWAPLAGTCGVLLL
SLVITLYCKRGRKKLLYIFKQPFMRPVQTTQEEDGCSCRFPEEEEGGCELRVKFSRSADAPAYKQGQNQLYNELNLG
RREEYDVLDKRRGRDPEMGGKPRRKNPQEGLYNELQKDKMAEAYSEIGMKGERRRGKGHDGLYQGLSTATKDTY
DALHMQALPPRGSGATNFSLLKQAGDVEENPGPMSLRGSLSRLLQTRVHSILKKSVHSVAVIGAPFSQGQKRKGV
EHGPAAIREAGLMKRLSSLGCHLKDFGDLSFTPVPKDDLYNNLIVNPRSVGLANQELAEVVSRAVSDGYSCVTLGG
DHSLAIGTISGHARHCPDLCVVWVDAHADINTPLTTSSGNLHGQPVSFLLRELQDKVPQLPGFSWIKPCISSASIVYI
GLRDVDPPEHFILKNYDIQYFSMRDIDRLGIQKVMERTFDLLIGKRQRPIHLSFDIDAFDPTLAPATGTPVVGGLTYR
EGMYIAEEIHNTGLLSALDLVEVNPQLATSEEEAKTTANLAVDVIASSFGQTREGGHIVYDQLPTPSSPDESENQAR
VRI
SEQ ID NO: 20
DNA seguence encoding the protein of SEQ ID NO: 19
ctcgagagctttggcgtaatcatggtcatagctgtttcctgtgtgaaattgttatccgctcacaattccacacaacatacga
gccggaagcataaagtgtaaagcctggggtgcctaatgagtgagctaactcacattaattgcgttgcgctcactgccc
gctttccagtcgggaaacctgtcgtgccagctgcattaatgaatcggccaacgcgcggggagaggcggtttgcgtatt
gggcgctcttccgcttcctcgctcactgactcgctgcgctcggtcgttcggctgcggcgagcggtatcagctcactcaa
aggcggtaatacggttatccacagaatcaggggataacgcaggaaagaacatgtgagcaaaaggccagcaaaagg
ccaggaaccgtaaaaaggccgcgttgctggcgtttttccataggctccgcccccctgacgagcatcacaaaaatcga
cgctcaagtcagaggtggcgaaacccgacaggactataaagataccaggcgtttccccctggaagctccctcgtgc
gctctcctgttccgaccctgccgcttaccggatacctgtccgcctttctcccttcgggaagcgtggcgctttctcaatgct
cacgctgtaggtatctcagttcggtgtaggtcgttcgctccaagctgggctgtgtgcacgaaccccccgttcagcccga
ccgctgcgccttatccggtaactatcgtcttgagtccaacccggtaagacacgacttatcgccactggcagcagccac
tggtaacaggattagcagagcgaggtatgtaggcggtgctacagagttcttgaagtggtggcctaactacggctacac
tagaaggacagtatttggtatctgcgctctgctgaagccagttaccttcggaaaaagagttggtagctcttgatccggca
aacaaaccaccgctggtagcggtggtttttttgtttgcaagcagcagattacgcgcagaaaaaaaggatctcaagaag
atcctttgatcttttctacggggtctgacgctcagtggaacgaaaactcacgttaagggattttggtcatgagattatcaaa
aaggatcttcacctagatccttttaaattaaaaatgaagttttaaatcaatctaaagtatatatgagtaaacttggtctgaca
gttaccaatgcttaatcagtgaggcacctatctcagcgatctgtctatttcgttcatccatagttgcctgactccccgtcgtg
tagataactacgatacgggagggcttaccatctggccccagtgctgcaatgataccgcgagacccacgctcaccggc
tccagatttatcagcaataaaccagccagccggaagggccgagcgcagaagtggtcctgcaactttatccgcctccat
ccagtctattaattgttgccgggaagctagagtaagtagttcgccagttaatagtttgcgcaacgttgttgccattgctgct
ggcatcgtggtgtcacgctcgtcgtttggtatggcttcattcagctccggttcccaacgatcaaggcgagttacatgatc
ccccatgttgtgcaaaaaagcggttagctccttcggtcctccgatcgttgtcagaagtaagttggccgcagtgttatcact
catggttatggcagcactgcataattctcttactgtcatgccatccgtaagatgcttttctgtgactggtgagtactcaacc
aagtcattctgagaatagtgtatgcggcgaccgagttgctcttgcccggcgtcaatacgggataataccgcgccacat
agcagaactttaaaagtgctcatcattggaaaacgttcttcggggcgaaaactctcaaggatcttaccgctgttgagatc
cagttcgatgtaacccactcgtgcacccaactgatcttcagcatcttttactttcaccagcgtttctgggtgagcaaaaac
aggaaggcaaaatgccgcaaaaaagggaataagggcgacacggaaatgttgaatactcatactcttcctttttcaatat
tattgaagcatttatcagggttattgtctcatgagcggatacatatttgaatgtatttagaaaaataaacaaataggggttcc
gcgcacatttccccgaaaagtgccacctgacgtctaagaaaccattattatcatgacattaacctataaaaataggcgta
tcacgaggccctttcgtcttcaagctgcctcgcgcgtttcggtgatgacggtgaaaacctctgacacatgcagctcccg
gagacggtcacagcttgtctgtaagcggatgccgggagcagacaagcccgtcagggcgcgtcagcgggtgttggc
gggtgtcggggcgcagccatgacccagtcacgtagcgatagttactatgcggcatcagagcagattgtactgagagt
gcaccatatgcggtgtgaaataccgcacagatgcgtaaggagaaaataccgcatcaggcgccattcgccattcaggc
tgcgcaactgttgggaagggcgatcggtgcgggcctcttcgctattacgccagctggcgaaagggggatgtgctgca
aggcgattaagttgggtaacgccagggttttcccagtcacgacgttgtaaaacgacggccagtgaattagtactctagc
ttaagtaacgccattttgcaaggcatggaaaatacataactgagaatagagaagttcagatcaaggttaggaacagag
agacagcagaatatgggccaaacaggatatctgtggtaagcagttcctgccccggctcagggccaagaacagttgg
aacagcagaatatgggccaaacaggatatctgtggtaagcagttcctgccccggctcagggccaagaacagatggt
ccccagatgcggtcccgccctcagcagtttctagagaaccatcagatgtttccagggtgccccaaggacctgaaatg
accctgtgccttatttgaactaaccaatcagttcgcttctcgcttctgttcgcgcgcttctgctccccgagctcaataaaag
agcccacaacccctcactcggcgcgccagtcctccgatagactgcgtcgcccgggtacccgtattcccaataaagcc
tcttgctgtttgcatccgaatcgtggactcgctgatccttgggagggtctcctcagattgattgactgcccacctcggggg
tctttcatttggaggttccaccgagatttggagacccctgcccagggaccaccgacccccccgccgggaggtaagct
ggccagcggtcgtttcgtgtctgtctctgtctttgtgcgtgtttgtgccggcatctaatgtttgcgcctgcgtctgtactagtt
ggctaactagatctgtatctggcggtcccgcggaagaactgacgagttcgtattcccggccgcagcccctgggagac
gtcccagcggcctcgggggcccgttttgtggcccattctgtatcagttaacctacccgagtcggactttttggagctccg
ccactgtccgaggggtacgtggctttgttgggggacgagagacagagacacttcccgcccccgtctgaatttttgcttt
cggttttacgccgaaaccgcgccgcgcgtcttgtctgctgcagcatcgttctgtgttgtctctgtctgactgtgtttctgtatt
tgtctgaaaattagctcgacaaagttaagtaatagtccctctctccaagctcacttacaggcggccgaattcgccgccg
ccatgcctcgcggctggacagccctgtgcctgctgtctctgctgccatccggcttcatgagcctggataataacggcacagccac
cccagagctgcctacacagggcaccttcagcaatgtgtccacaaacgtgagctatcaggagaccacaaccccttctaccctgg
gatccacaagcctgcaccccgtgtctcagcacggcaacgaagccaccaccaacatcaccgagaccacagtgaagtttacctc
cacctctgtgattacctctgtgtacggaaatacaaactccagcgtgcagtctcagacatctgtgatctccacagtgtttacaacac
ctgccaatgtgtccaccccagagacaaccctgaagcccagcctgtctcctggaaatgtgtccgatctgtctaccacctccaccagcc
tggccacctctcccaccaagccctatacctcctcttctcccatcctgagcgatatcaaagccgagatcaaatgcagcgggattcgg
gaagtgaaactgacacagggcatctgcctggaacagaataagacatccagctgcgccgagtttaagaaagatagaggagag
ggactggccagggtgctgtgtggcgaagagcaggccgacgccgatgccggcgcccaggtgtgttccctgctgctggcccagtc
tgaggtgcgcccccagtgcctgctgctggtgctggccaatcggacagaaattagcagcaagctgcagctgatgaaaaaacacc
agagcgatctgaaaaagctgggcatcctggactttaccgagcaggacgtggcctctcaccagagctacagccagaaaacact
gatcgccctggtgaccagcggagccctgctggccgtgctgggcatcaccggatatttcctgatgaataggcgcagctggagccc
caccggcgaacggctggagctggagcctgtcgaccgagtgaagcagaccctgaactttgatctgctgaagctggccggcgac
gtggagtccaaccccgggccagggaatATGGCCTTACCAGTGACCGCCTTGCTCCTGCCGCTGGC
CTTGCTGCTCCACGCCGCCAGGCCGGATATTCTGCTCACACAGACCCCACTCTCCCTG
CCCGTGTCACTCGGGGATCAGGCTAGCATTTCTTGCCGCTCATCTCAGTCTCTGGTCC
ACCGGAATGGGAACACATACCTCCATTGGTACCTCCAGAAACCTGGACAGAGCCCTAA
ACTGCTCATCCACAAAGTCTCAAATCGGTTCTCCGGCGTGCCCGATCGCTTTAGCGGAT
CCGGATCTGGGACCGACTTCACACTGAAAATCTCACGAGTGGAGGCTGAGGATCTCGG
CGTCTACTTCTGTAGTCAGAGTACCCACGTCCCACCCCTCACCTTTGGCGCTGGAACAA
AACTGGAGCTGAAACGAGCCGATGCTGCTCCTACCGTGTCCATCTTTCCTGGCTCCGG
GGGAGGCGGGAGCGGAGGCGAAGTGAAACTCCAGCAGTCTGGCCCTTCTCTCGTGGA
ACCTGGCGCTTCTGTGATGATCTCCTGTAAGGCCTCTGGATCTTCCTTTACCGGCTACA
ACATGAACTGGGTCCGGCAGAACATTGGCAAATCCCTGGAATGGATTGGCGCCATCGA
TCCTTACTACGGCGGCACATCATACAATCAGAAATTCAAGGGGCGAGCAACACTCACTG
TCGACAAATCTTCATCCACCGCCTACATGCACCTGAAATCTCTCACATCCGAGGATAGT
GCTGTCTACTACTGTGTCTCTGGCATGGAATACTGGGGACAGGGAACTTCTGTCACCG
TGTCTAGTGCCAAAACCACACCTCCCTCCGTGTACGGACGAGTCACTGTCTCATCTGCT
GAACCAAAATCCTGTGACAAAACACACACATGCCCACCTTGTCCTGCCCCTGAACTGCT
CGGCGGACCTTCCGTCTTTCTGTTTCCCCCCAAACCCAAGGATACACTCATGATTTCTA
GGACCCCCGAAGTCACTTGTGTCGTGGTCGATGTGTCTCACGAGGATCCTGAAGTGAA
ATTCAACTGGTACGTGGACGGAGTCGAGGTCCACAATGCCAAAACAAAACCCCGGGAG
GAACAGTACAATAGCACCTACCGAGTCGTGTCCGTGCTCACCGTCCTCCATCAGGATT
GGCTGAACGGCAAAGAGTACAAGTGTAAAGTGAGTAACAAGGCTCTCCCCGCTCCTAT
TGAAAAAACCATCTCAAAAGCAAAAGGCCAGCCTAGGGAGCCTCAGGTCTACACACTG
CCACCCTCACGGGACGAACTCACCAAAAATCAGGTGTCCCTCACTTGCCTGGTGAAAG
GCTTCTACCCTTCCGATATCGCTGTGGAATGGGAGTCAAATGGGCAGCCCGAAAACAA
CTACAAAACAACCCCCCCTGTGCTCGATTCCGATGGCTCTTTTTTCCTGTACTCAAACT
CACCGTGGACAAATCACGCTGGCAGCAGGGGAATGTCTTTTCTTGCTCCGTGATGCAC
GAGGCCCTCCACAATCATTACACCCAGAAATCCCTCTCACTCTCACCCGGCAAAAAGGA
CCCTAAAACCACGACGCCAGCACCGCGACCACCAACACCGGCGCCAACCATCGCATC
GCAGCCCCTGTCCCTGCGCCCAGAGGCGTGCCGACCAGCGGCGGGGGGCGCAGTGC
ACACGAGGGGGCTGGACTTCGCCTGTGATATCTACATCTGGGCGCCCTTGGCCGGGA
CTTGTGGGGTCCTTCTCCTGTCACTGGTTATCACCCTTTACTGCAAACGGGGCAGAAAG
AAACTCCTGTATATATTCAAACAACCATTTATGAGACCAGTACAAACTACTCAAGAGGAA
GATGGCTGTAGCTGCCGATTTCCAGAAGAAGAAGAAGGAGGATGTGAACTGAGAGTGA
AGTTCAGCAGGAGCGCAGACGCCCCCGCGTACAAGCAGGGCCAGAACCAGCTCTATA
ACGAGCTCAATCTAGGACGAAGAGAGGAGTACGATGTTTTGGACAAGAGACGTGGC
CGGGACCCTGAGATGGGGGGAAAGCCGAGAAGGAAGAACCCTCAGGAAGGCCTGTAC
AATGAACTGCAGAAAGATAAGATGGCGGAGGCCTACAGTGAGATTGGGATGAAAGGCG
AGCGCCGGAGGGGCAAGGGGCACGATGGCCTTTACCAGGGTCTCAGTACAGCCACCA
AGGACACCTACGACGCCCTTCACATGCAGGCCCTGCCCCCTCGCGGCAGCGGCGCCA
CCAACTTCAGCCTGCTGAAGCAGGCCGGCGACGTGGAGGAAAACCCTGGCCCCATGT
CTTTAAGAGGCTCTTTATCTCGTCTGCTGCAGACICGIGIGCACAGCAIIIIAAAAAAGA
GCGTGCACAGCGTGGCCGTCATTGGAGCCCCCTTCAGCCAAGGCCAGAAGAGAAAGG
GCGTCGAACACGGACCCGCCGCCATCAGAGAAGCTGGTTTAATGAAGAGACTGAGCTC
TTTAGGCTGCCATTTAAAAGACTTGGAGATTTGTCTTTTACCCCCGTCCCCAAGGACG
ATTTATACAATAATTTAATCGTGAACCCCAGATCCGTGGGACTGGCTAACCAAGAACTG
GCCGAGGTCGTGAGCAGAGCCGTGTCCGACGGCTACTCTTGTGTGACTTTAGGCGGC
GATCACTCTTTAGCCATTGGCACAATCTCCGGACACGCTAGGCACTGCCCCGATTTATG
CGTGGTGTGGGTGGACGCTCACGCCGACATCAATACCCCTCTGACCACCAGCAGCGG
CAATTTACACGGACAGCCCGTCAGCTTTTTACTGAGGGAGCTGCAAGATAAGGTGCCT
CAGCTGCCCGGCTTCAGCTGGATCAAGCCTTGTATCAGCAGCGCTTCCATCGTGTACA
TTGGTTTAAGAGACGTGGACCCTCCCGAACACTTCATCCTCAAGAACTACGACATTCAG
TACTTCAGCATGAGGGATATCGATCGTCTCGGAATCCAGAAGGTGATGGAAAGGACCT
TCGATTTACTCATCGGAAAGAGGCAGAGGCCTATCCATTTATCCTTCGACATCGACGCC
TTCGATCCTACACTGGCCCCCGCTACTGGTACACCCGTTGTGGGCGGTTTAACCTATA
GGGAGGGCATGTACATCGCCGAAGAGATCCACAACACCGGTTTACTGAGCGCTCTGGA
TTTAGTGGAGGTGAATCCTCAGCTGGCCACCTCCGAGGAGGAGGCCAAAACCACCGC
CAATCTGGCCGTGGACGTGATCGCCAGCTCCTTCGGCCAGACCAGAGAGGGCGGCCA
CATTGTGTACGACCAGCTGCCCACACCCAGCTCCCCCGATGAGTCCGAAAACCAAGCT
CGTGTCAGAATCTGATAATAACCCAAGCTTaacacgagccatagatagaataaaagattttatttagtct
ccagaaaaaggggggaatgaaagaccccacctgtaggtttggcaagctagcttaagtaacgccattttgcaaggcat
ggaaaatacataactgagaatagagaagttcagatcaaggttaggaacagagagacagcagaatatgggccaaaca
ggatatctgtggtaagcagttcctgccccggctcagggccaagaacagttggaacagcagaatatgggccaaacag
gatatctgtggtaagcagttcctgccccggctcagggccaagaacagatggtccccagatgcggtcccgccctcagc
agtttctagagaaccatcagatgtttccagggtgccccaaggacctgaaatgaccctgtgccttatttgaactaaccaat
cagttcgcttctcgcttctgttcgcgcgcttctgctccccgagctcaataaaagagcccacaacccctcactcggcgcg
ccagtcctccgatagactgcgtcgcccgggtacccgtgttctcaataaaccctcttgcagttgcatccgactcgtggtct
cgctgttccttgggagggtctcctctgagtgattgactgcccacctcgggggtctttcatt

1. A fusion target-binding protein comprising a target binding moiety, an intracellular signalling region, and an arginase domain.

2. A fusion target-binding protein according to claim 1 that is a chimeric antigen receptor (CAR).

3. A fusion target-binding protein according to claim 1 or claim 2, wherein the arginase domain comprises the arginase type I enzyme, or a fragment or variant thereof.

4. A fusion target-binding protein according to claim 3, wherein the fragment of the arginase type I domain comprises at least 75% of the amino acid sequence of wild type arginase type I.

5. A fusion target-binding protein according to claim 3, wherein the arginase type I variant shares at least 75% sequence identity with the corresponding portion of arginase type I.

6. A fusion target-binding protein according to claim 1 or claim 2, wherein the arginase domain comprises the arginase type II enzyme, or a fragment or variant thereof.

7. A fusion target-binding protein according to claim 6, wherein the fragment of the arginase type I domain comprises at least 75% of the amino acid sequence of wild type arginase type II.

8. A fusion target-binding protein according to claim 6, wherein the arginase type II variant shares at least 75% sequence identity with the corresponding portion of arginase type II.

9. A fusion target-binding protein according to any preceding claim, comprising an intracellular signalling region selected from the group consisting of: a 4-1BB signalling region; an OX-40 signalling region; a CD28 signalling region; an ICOS signalling region; and a CD3 ζ signalling region.

10. A fusion target-binding protein according to any preceding claim, comprising a target binding domain that binds to at least one antigen selected from the group consisting of: GD2; CD33; Mesothelin; EGFRvIII; VEGFR2; FAP; EpCam; GPC3; CD133; IL13Ra; EphA2; Muc1; BCMA; CD70; CD123; ROR1; PSMA; CD5; GAP; CEA; PSCA; Her2; and CD19.

11. A fusion target-binding protein according to any preceding claim, comprising an target binding domain selected from the group consisting of: an antibody; an antibody fragment (such as an scFv); a variant of an antibody or antibody fragment; a TCR, such as a TCR α chain or a TCR β chain; and an aptamer.

12. A fusion target-binding protein according to any preceding claim, comprising a target binding domain that binds GD2.

13. A fusion target-binding protein according to any of claims 1 to 11, comprising a target binding domain that binds CD33.

14. A fusion target-binding protein according to any of claims 1 to 11, comprising a target binding domain that binds mesothelin.

15. A fusion target-binding protein according to any of claims 1 to 11, comprising a target binding domain that binds EGFRvIII.

16. A cell comprising a fusion target-binding protein according to any preceding claim.

17. A cell according to claim 16 for use as a medicament.

18. A cell for use according to claim 17 in autologous treatment.

19. A cell for use according to claim 18 in heterologous treatment.

20. A cell according to any of claims 16 to 19, selected from the group consisting of: a T cell; and a natural killer (NK) cell.

21. A T cell according to claim 20.

22. A nucleic acid molecule encoding a fusion target-binding protein according to any of claims 1 to 15.

23. A pharmaceutical composition comprising a fusion target-binding protein according to any of claims 1 to 15, a cell according to any of claims 16 to 21, or a nucleic acid molecule according to claim 22, and a pharmaceutically acceptable carrier or diluent.

24. A fusion target-binding protein according to any of claims 1 to 15, a cell according to any of claims 16 to 21, or a nucleic acid molecule according to claim 22, or a pharmaceutical composition according to claim 20, for use in the prevention and/or treatment of cancer.

25. A cell comprising a fusion target-binding protein according to claim 12, for use as a medicament in the prevention and/or treatment of a disease associated with expression of GD2, such as neuroblastoma.

26. A cell comprising a fusion target-binding protein according to claim 13, for use as a medicament in the prevention and/or treatment of a disease associated with expression of CD33, such as acute myeloid leukaemia.

27. A cell comprising a fusion target-binding protein according to claim 14, for use as a medicament in the prevention and/or treatment of a disease associated with expression of mesothelin, such as a cancer selected from the group consisting of: mesothelioma; ovarian cancer; and pancreatic cancer.

28. A cell comprising a fusion target-binding protein according to claim 15, for use as a medicament in the prevention and/or treatment of a disease associated with expression of EGFRvIII, such as glioblastoma.

29. A cell for use according to any of claims 25 to 28 in autologous treatment.

30. A cell for use according to any of claims 25 to 28 in heterologous treatment.

31. A method of manufacturing a cell according to any of claims 16 to 21, the method comprising providing a cell with a nucleic acid molecule according to claim 22, such that the nucleic acid molecule is expressed by the cell to produce a fusion target-binding protein according to any of claims 1 to 15.

32. A method of preventing and/or treating a disease, the method comprising providing a protein according to any of claims 1 to 15, a cell according to any of claims 16 to 21, or a nucleic acid molecule according to claim 22, to a subject in need of such prevention and/or treatment.

33. A method according to claim 32, wherein the protein, cell or nucleic acid is provided by a pharmaceutical composition according to claim 23.

34. A method according to claim 32 or claim 33, where the disease to be prevented and/or treated is cancer.

35. A method according to claim 34, wherein the cancer is selected from the group consisting of: neuroblastoma; acute myeloid leukaemia (AML); mesothelioma; ovarian cancer; pancreatic cancer; and glioblastoma.
